(19) United States
(12) Patent Application Publication
(10) Pub. No.: US 2026/0278892 A1
(43) Pub. Date: Sep. 17, 2026

(54) GENERALIZED IMAGE RESTORATION WITH SYNTHETIC PRIOR FOR SPARSE-VIEW COMPUTED TOMOGRAPHY

(71) Applicant: CARL ZEISS X-RAY MICROSCOPY, INC., Dublin, CA (US)

(72) Inventor: Matthew ANDREW, Dublin, CA (US)

(21) Appl. No.: 19/668,788

(22) Filed: May 5, 2026

Related U.S. Application Data
(63) Continuation-in-part of application No. PCT/US2025/014522, filed on Feb. 5, 2025.
(60) Provisional application No. 63/549,818, filed on Feb. 5, 2024.

(51) Int. Cl. G06T 12/20; G06T 5/10; G06T 7/12; G06T 15/08; G06V 10/82; G16H 30/40

(52) U.S. Cl. CPC G06T 12/20; G06T 5/10; G06T 7/12; G06T 15/08; G06V 10/82; G16H 30/40

(57) ABSTRACT

A computer-implemented method is designed for reconstructing an image from sparse-view computed tomography (CT) imaging data. The sparse-view CT imaging data is acquired at a throughput-conditioned acquisition setting. The sparse-view imaging data is reconstructed using an analytical reconstruction algorithm. A final reconstructed volume is generated from a trained image quality model created using a synthetic prior. A throughput-conditioned spectral noise field is constructed by computing a throughput difference field. A throughput-conditioned synthesized noise volume is generated by adding the spectral noise field to the final reconstructed volume. A throughput-conditioned machine learning model is trained using the synthesized noise volume as input and the final reconstructed volume as target. The throughput-conditioned machine learning model is applied to the sparse-view reconstruction to generate a throughput-conditioned reconstructed volume.

This application is a continuation-in-part of International Patent Application No. PCT/US2025/014522, filed on Feb. 5, 2025, under the Patent Cooperation Treaty, which was published as International Publication No. WO 2025/170852 A1 (hereinafter, the “Parent PCT Application:). This application claims the benefit of priority to the Parent PCT Application under 35 U.S.C. § 365(c) and 35 U.S.C. §§ 119(a)-(d), as applicable, designating the United States.

This application is a continuation-in-part of the Parent PCT Application in that it includes subject matter disclosed in the Parent PCT Application and further adds new matter not disclosed therein. To the extent that any claims presented herein are supported by the disclosure of the Parent PCT Application, those claims are entitled to the benefit of the filing date of the Parent PCT Application. Claims directed to new matter added in this continuation-in-part application are entitled to the filing date of this application.

The present disclosure relates to image restoration generally and more specifically to restoration of radiological imaging data associated with sparse-view computed tomography, such as X-ray microscopy and computed tomography (CT) imaging data.

### BACKGROUND

X-ray Microscopy Imaging is a field of imaging that is used to acquire imaging data for many different types of samples across many different use cases. X-ray Microscopy Imaging has found uses in biology (e.g., imaging biomaterials, soft tissues, and the like), material science (e.g., imaging the internal microstructure of a material), manufacturing (e.g., non-destructively imaging internal components), and many other fields. Individual images (e.g., projections) can be acquired by directing radiation from an X-ray source, through a sample, towards a detector. Multiple projections can be acquired for a single sample by rotating the direction of travel of the X-ray radiation with respect to the sample (e.g., rotating the sample with respect to the X-ray source and detector). Often, the acquired imaging data (e.g., containing multiple projections) is used to generate a three dimensional reconstructed volumes of the sample that was imaged, such as through the use of computed tomography (CT).

While X-ray imaging provides many benefits, it often suffers from a range of artifacts and imaging issues, including noise, sparse sampling artifacts, beam hardening, missing angle artifacts, and other issues. Many of these issues have historically been dealt with using a combination of specialized 3D reconstruction technology and post processing filters. These techniques, however, give suboptimal imaging results and can introduce new artifacts or biases in the results that should be avoided if possible.

In many practical settings, it is desirable to reduce the number of projection views acquired during a CT scan. Reducing the number of projection views is referred to herein as sparse-view acquisition. It can increase scan throughput, reduce total scan time, and lower the radiation dose delivered to the sample. However, sparse-view acquisition introduces significant imaging artifacts. For example, streak artifacts and noise amplification associated with sparse-view acquisition degrade image quality and limit the effectiveness of downstream analysis.

Existing approaches to image reconstruction from sparse-view CT data have significant limitations. Conventional analytical reconstruction algorithms, such as filtered back-projection, produce reconstructions with severe noise and artifacts when applied to sparse-view data. Some deep learning-based methods have been developed for sparse-view CT reconstruction. But these methods generally focus on two-dimensional fan-beam CT or medical imaging applications. Such methods do not robustly address the combined challenges of noise reduction and sparse sampling artifact removal in three-dimensional cone-beam CT for industrial or high-throughput settings.

Other approaches are effective for noise and artifact removal in standard full-view CT acquisitions but are not optimized for scenarios with sparse projection data. These methods train deep learning models on full-view data and do not account for the unique noise characteristics and artifact patterns introduced by sparse-view acquisition.

Accordingly, there is a need for improved methods and systems for reconstructing CT images from sparse-view data that reduce noise and artifacts while maintaining high image quality in industrial and high-throughput imaging applications.

### BRIEF SUMMARY

The term embodiment and like terms are intended to refer broadly to all of the subject matter of this disclosure and the claims below. Statements containing these terms should be understood not to limit the subject matter described herein or to limit the meaning or scope of the claims below. Embodiments of the present disclosure covered herein are defined by the claims below, supplemented by this summary. This summary is a high-level overview of various aspects of the disclosure and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification of this disclosure, any or all drawings and each claim.

Embodiments of the present disclosure include a computer-implemented image restoration method, which includes receiving imaging data associated with a subject, generating a synthetic prior by applying a first imaging restoration process to the received imaging data, determining a forward imaging operator, generating synthetic imaging data by applying the forward imaging operator to the synthetic prior, training a second imaging restoration process based at least in part on the synthetic imaging data and the synthetic prior, and applying the second imaging restoration process to the received imaging data to generate restored imaging data. The image restoration method may also include where applying the first imaging restoration process includes applying a trained machine learning algorithm to the received imaging data. The image restoration method may also include where the second imaging restoration process includes a machine learning algorithm, and where training the second imaging restoration process includes optimizing a hyperparameter of the machine learning algorithm. The image restoration method may also include where determining the forward imaging operator includes receiving metadata associated with the imaging data, and selecting the forward imaging operator from a set of preset forward imaging operators based at least in part on the received metadata. The image restoration method may also include where the received imaging data is in the image space and the synthetic prior is in the image space. The image restoration method may also include where determining the forward imaging operator includes calculating an initial noise distribution based at least in part on the received imaging data and the synthetic prior, calculating one or more parameters associated with the initial noise distribution, and generating a synthetic noise field based at least in part on the calculated one or more parameters, where generating the synthetic imaging data includes applying the synthetic noise field to the synthetic prior. The image restoration method may also include where training the second imaging restoration process includes determining one or more regions of high gradient associated with the synthetic prior, and training a machine learning algorithm using a loss function based at least in part on the synthetic prior and the synthetic imaging data, where the loss function weights a loss in the determined one or more regions of high gradient higher than a loss not in the determined one or more regions of high gradient. The image restoration method may also include where the received imaging data is in the projection space and the synthetic prior is in the image space. The image restoration method may also include where generating the synthetic prior includes generating an initial reconstruction based at least in part on the received imaging data, determining an estimated noise field based at least in part on the initial reconstruction, determining an available angle range associated with the received imaging data, accessing a training volume, forward-projecting the training volume in the available angle range to generate constrained training volume imaging data, forward-projecting the training volume in full range to generate full training volume imaging data, generating a constrained training reconstruction based at least in part on the constrained training volume imaging data, generating a full training reconstruction based at least in part on the full training volume imaging data, updating the constrained training reconstruction by adding the estimated noise field to the constrained training reconstruction, training the first imaging restoration process based at least in part on the constrained training reconstruction and the full training reconstruction, and applying the trained first imaging restoration process to the initial reconstruction to obtain the synthetic prior. The image restoration method may also further include outputting the restored imaging data using a display device.

A system includes a control system including one or more processors, and a memory having stored thereon machine readable instructions, where the control system is coupled to the memory, and the method of any one of claims 1 to 17 is implemented when the machine executable instructions in the memory are executed by at least one of the one or more processors of the control system.

A computer program product tangibly embodied in a non-transitory machine-readable storage medium, the computer program product includes instructions which, when executed by a computer, cause the computer to carry out the method of any one of claims 1 to 17.

The image restoration method may also include where optimizing the hyperparameter includes generating interim restored imaging data by applying the second imaging restoration process to the synthetic imaging data, and determining a difference between the synthetic prior and the interim restored imaging data, where the hyperparameter is optimized when the difference between the synthetic prior and the interim restored imaging data is minimized. The image restoration method may also include where calculating the initial noise distribution includes including applying the equation Nᵢ=D−Sₐ where Nᵢ is the initial noise distribution, D is the received imaging data, and Sₐ is the synthetic prior. The image restoration method may also include where calculating the one or more parameters associated with the initial noise distribution includes generating a frequency domain representation of the initial noise distribution, and where generating the synthetic noise field includes generating a magnitude field from the frequency domain representation of the initial noise distribution, and creating the synthetic noise field based at least in part on the magnitude field. The image restoration method may also include where generating the synthetic noise field further includes generating a random phase field using a uniform random number generator, and where creating the synthetic noise field is further based at least in part on the random phase field. The image restoration method may also include where calculating the one or more parameters associated with the initial noise distribution further includes calculating a local spatially variant standard deviation of the initial noise distribution, and where generating the synthetic noise field further includes generating a random noise field based at least in part on the local spatially variant standard deviation, and generating a phase field based at least in part on a frequency domain representation of the random noise field, and where creating the synthetic noise field is further based at least in part on the generated phase field. The image restoration method may also include where generating the synthetic noise field includes applying the equation N=real(ifft(N_{imag}×cos(Gₚₕₐₛₑ)+N_{imag}×sin(Gₚₕₐₛₑ)i)), where N is the synthetic noise field, N_{imag} is the magnitude field of the frequency domain representation of the initial noise distribution, Gₚₕₐₛₑ is a phase field derived from a frequency domain representation of a random noise field generated based at least in part on a calculated local spatially variant standard deviation of the initial noise distribution, i is \sqrt{−1}, real is a real component function, and ifft is an inverse Fourier transform. The image restoration method may also include where the synthetic noise field has equivalent local Fourier magnitudes to the initial noise distribution, and where local phases of the synthetic noise field are fully decorrelated to the initial noise distribution. The image restoration method may also include where generating the synthetic imaging data includes generating a new projection dataset by forward-projecting the synthetic prior through a projective geometry corresponding to the received imaging data, determining an outside-field-of-view (OFOV) attenuation representation based at least in part on the new projection dataset and the received imaging data, the OFOV attenuation representation corresponding to material outside a field of view of the received imaging data, denoising the OFOV attenuation representation, generating an updated new projection dataset by combining the denoised OFOV attenuation representation and the new projection dataset, and generating the synthetic imaging data by adding the estimated noise field to back-projection of the updated new projection dataset.

The image restoration method may also further include outputting the restored imaging data using a display device. The image restoration method may also include where the received imaging data is in a projection domain. The image restoration method may also include where the synthetic prior is in an image domain, the first imaging restoration process usable to generate reconstructed datasets (e.g., reconstructed volume datasets) from projection datasets. The image restoration method may also include where the received imaging data includes a projection dataset acquired via an X-ray imaging device. The image restoration method may also include where the received imaging data is in an image domain and the synthetic prior is in the image domain. The image restoration method may also include where the received imaging data is representative of a plurality of projections acquired at a plurality of different angles with respect to the subject. The image restoration method may also include where the received imaging data is associated with a transmission imaging study of the subject. The image restoration method may also include where the transmission imaging study is an X-ray imaging study.

Embodiments of the present disclosure include a computer-implemented method for reconstructing an image from sparse-view computed tomography (CT) imaging data. The sparse-view imaging data of a subject is acquired by a CT imaging device at a throughput-conditioned acquisition setting. The sparse-view imaging data has fewer projection views than full-view imaging data of the subject. The sparse-view imaging data is reconstructed using an analytical reconstruction algorithm to produce a sparse-view reconstruction. A final reconstructed volume of the subject is then generated from a trained image quality model that is created using a synthetic prior. Additionally, a throughput-conditioned spectral noise field is generated by computing a throughput difference field between the sparse-view reconstruction and the final reconstructed volume. The throughput difference field is transformed into a throughput frequency domain to obtain a throughput magnitude spectrum. The throughput magnitude spectrum is combined with a throughput phase field to produce a synthesized throughput frequency-domain representation. And an inverse frequency-domain transform is applied to the synthesized throughput frequency-domain representation to obtain the throughput-conditioned spectral noise field. Moreover, the method includes generating a throughput-conditioned synthesized noise volume by adding the throughput-conditioned spectral noise field to the final reconstructed volume. Further, a throughput-conditioned machine learning model is trained using the throughput-conditioned synthesized noise volume as input and the final reconstructed volume as a target. The method includes applying the throughput-conditioned machine learning model to the sparse-view reconstruction to generate a throughput-conditioned reconstructed volume.

In some embodiments, the trained image quality model is created by receiving the full-view imaging data of the subject acquired by the CT imaging device. The full-view imaging data is partitioned into a first projection subset and a second projection subset. The first projection subset and the second projection subset are independently reconstructed using the analytical reconstruction algorithm to produce a first grouped reconstruction and a second grouped reconstruction, respectively. Then, a first machine learning model is trained on the first grouped reconstruction and the second grouped reconstruction to learn a synthetic prior model. The first machine learning model is applied to a baseline reconstruction derived from the full-view imaging data to generate a synthetic prior. A full-view spectral noise field is constructed by computing a full-view difference field between the baseline reconstruction and the synthetic prior. The full-view difference field is transformed into a full-view frequency domain to obtain a full-view magnitude spectrum. The full-view magnitude spectrum is combined with a full-view phase field to produce a synthesized full-view frequency-domain representation. By applying an inverse frequency-domain transform to the synthesized full-view frequency-domain representation, the full-view spectral noise field is obtained. Additionally, a synthesized full-view noise volume is generated by adding the full-view spectral noise field to the synthetic prior. A second machine learning model is trained using the synthesized full-view noise volume as input and the synthetic prior as a target to produce the trained image quality model.

In some embodiments, the method further includes providing a residual neural network to the first machine learning model. Using the residual neural network, an output of the first machine learning model is generated based on a learned correction. The output is expressed as

$$\hat{y} = x + {f}_{θ} ( x ) ,$$

wherein x is an input reconstruction and f_{θ}(x) is the learned correction.

In some embodiments, the residual neural network further includes an initial three-dimensional convolution layer that extracts inter-slice features from a stack of adjacent input slices. Additionally, the residual neural network includes a two-dimensional encoder-decoder network that processes the extracted features at multiple resolution levels. Further, the residual neural network includes a final three-dimensional output convolution layer that produces a volumetric output.

In some embodiments, the analytical reconstruction algorithm comprises a filtered back-projection (FBP). Additionally, the filtered back-projection is used to generate each of the baseline reconstruction, the first grouped reconstruction, the second grouped reconstruction, and the sparse-view reconstruction.

In some embodiments, constructing the full-view spectral noise field further includes partitioning the full-view difference field into local volumetric chunks. For each chunk, a local fast Fourier transform (FFT) is computed to generate a local magnitude spectrum. Additionally, for each chunk, the full-view phase field is generated by one of generating randomized phases using a uniform random number generator or computing an auto-phase field derived from a frequency-domain representation of a random noise field seeded by a local spatially variant standard deviation of the full-view difference field. Further, for each chunk, the local magnitude spectrum is combined with the full-view phase field and applying an inverse FFT to produce a local synthesized noise chunk.

In some embodiments, training the second machine learning model includes computing a mean and a standard deviation of the synthesized full-view noise volume. Additionally, both the synthesized full-view noise volume and the synthetic prior are normalized using the mean and standard deviation prior to training the second machine learning model. Normalized values are computed as

$${x}^{′} = \frac{x - μ}{σ} , {y}^{′} = \frac{y - μ}{σ} ,$$

where μ is the mean and σ is the standard deviation of the synthesized full-view noise volume.

In some embodiments, training the second machine learning model further includes deriving an edge mask from the synthetic prior by computing a magnitude of a Gaussian-smoothed residual of the synthetic prior. Moreover, a loss function is weighed during training. Further, regions identified by the edge mask receive higher loss weights than non-edge regions.

In some embodiments, deriving the edge mask further comprises restricting the edge mask to a disk-shaped support region when a circular field-of-view constraint is active.

In some embodiments, constructing the throughput-conditioned spectral noise field further includes partitioning the throughput difference field into local volumetric chunks. For each chunk, a local fast Fourier transform (FFT) is computed to generate a local magnitude spectrum. Additionally, for each chunk, the throughput phase field is generated by one of: (i) generating randomized phases using a uniform random number generator, or (ii) computing an auto-phase field derived from a frequency-domain representation of a random noise field seeded by a local spatially variant standard deviation of the throughput difference field. Further, for each chunk, the local magnitude spectrum is combined with the throughput phase field and applying an inverse FFT to produce a local synthesized noise chunk.

In some embodiments, each local volumetric chunk has dimensions of 64×64×64 voxels. The throughput difference field is padded along a slice direction to a multiple of 64 voxels prior to partitioning.

In some embodiments, the method further includes rescaling the local synthesized noise chunk. A standard deviation of each of the local synthesized noise chunk matches a standard deviation of a corresponding chunk of the throughput difference field.

In some embodiments, training the throughput-conditioned machine learning model further includes a mean-squared error loss defined as

$${L}_{MSE} = \frac{1}{N} {\sum }_{i = 1}^{N} {( {\hat{y}}_{i} - {y}_{i} )}^{2} ,$$

where ŷᵢ is a predicted output and yᵢ is a corresponding target.

In some embodiments, the throughput-conditioned acquisition setting defines a reduction factor in a number of projection views relative to the full-view imaging data. The full-view imaging data is grouped according to the throughput-conditioned acquisition setting to select a subset of projection views corresponding to the sparse-view imaging data.

In some embodiments, the CT imaging device acquires the sparse-view imaging data using a three-dimensional cone-beam CT geometry.

In some embodiments, the subject comprises an industrial sample, and the sparse-view imaging data is acquired in a high-throughput industrial inspection setting.

Embodiments of the present disclosure also include a system for reconstructing computed tomography (CT) images from sparse-view imaging data. The system includes a CT imaging device having a throughput-conditioned acquisition setting, one or more processors, and a memory. The memory is coupled to the one or more processors and stores instructions. When executed by the one or more processors, the instructions cause the system to receive sparse-view imaging data of a subject acquired by the CT imaging device at the throughput-conditioned acquisition setting. The sparse-view imaging data has fewer projection views than full-view imaging data of the subject. Additionally, the instructions cause the system to reconstruct the sparse-view imaging data using an analytical reconstruction algorithm to produce a sparse-view reconstruction. The instructions further cause the system to generate a final reconstructed volume of the subject. The final reconstructed volume is generated from a trained image quality model that is created using a synthetic prior. Moreover, the instructions cause the system to construct a throughput-conditioned spectral noise field by computing a throughput difference field between the sparse-view reconstruction and the final reconstructed volume. The throughput difference field is transformed into a throughput frequency domain to obtain a throughput magnitude spectrum, combining the throughput magnitude spectrum with a throughput phase field to produce a synthesized throughput frequency-domain representation. Additionally, an inverse frequency-domain transform is applied to the synthesized throughput frequency-domain representation to obtain the throughput-conditioned spectral noise field. The instructions cause the system to generate a throughput-conditioned synthesized noise volume by adding the throughput-conditioned spectral noise field to the final reconstructed volume. A throughput-conditioned machine learning model is trained using the throughput-conditioned synthesized noise volume as input and the final reconstructed volume as a target. Further, the throughput-conditioned machine learning model is applied to the sparse-view reconstruction to generate a throughput-conditioned reconstructed volume.

In some embodiments of the system, the instructions further cause the system to generate the final reconstructed volume by receiving the full-view imaging data. The full-view imaging data is partitioned into two projection subsets. Each subset is independently reconstructed using the analytical reconstruction algorithm to produce a pair of grouped reconstructions. A first residual neural network is trained on the pair of grouped reconstructions to learn a synthetic prior model. The first residual neural network is applied to a full-data reconstruction to generate the synthetic prior. Moreover, a full-view spectral noise field is constructed by computing a full-view difference field between the full-data reconstruction and the synthetic prior. Further, the full-view difference field is transformed into a frequency domain to obtain a full-view magnitude spectrum. The full-view magnitude spectrum is combined with a full-view phase field to produce a synthesized full-view frequency-domain representation. Moreover, an inverse frequency-domain transform is applied to obtain the full-view spectral noise field. The trained image quality model is applied to the full-data reconstruction to generate the final reconstructed volume.

Embodiments of the present disclosure further include a non-transitory computer-readable medium storing instructions. When executed by one or more processors, the instructions cause the one or more processors to perform a method. The method includes receiving sparse-view imaging data. The sparse-view imaging data of a subject is acquired by a CT imaging device at a throughput-conditioned acquisition setting. The sparse-view imaging data has fewer projection views than full-view imaging data of the subject. The sparse-view imaging data is reconstructed using an analytical reconstruction algorithm to produce a sparse-view reconstruction. A final reconstructed volume of the subject is then generated from a trained image quality model that is created using a synthetic prior. Additionally, a throughput-conditioned spectral noise field is generated by computing a throughput difference field between the sparse-view reconstruction and the final reconstructed volume. The throughput difference field is transformed into a throughput frequency domain to obtain a throughput magnitude spectrum. The throughput magnitude spectrum is combined with a throughput phase field to produce a synthesized throughput frequency-domain representation. And an inverse frequency-domain transform is applied to the synthesized throughput frequency-domain representation to obtain the throughput-conditioned spectral noise field. Moreover, the method includes generating a throughput-conditioned synthesized noise volume by adding the throughput-conditioned spectral noise field to the final reconstructed volume. Further, a throughput-conditioned machine learning model is trained using the throughput-conditioned synthesized noise volume as input and the final reconstructed volume as a target. The method includes applying the throughput-conditioned machine learning model to the sparse-view reconstruction to generate a throughput-conditioned reconstructed volume.

In some embodiments of the non-transitory computer-readable medium, the analytical reconstruction algorithm comprises filtered back-projection. The subject is imaged using a three-dimensional cone-beam CT geometry in an industrial or high-throughput imaging application.

Other technical features may be readily apparent to one skilled in the art from the following figures, descriptions, and claims.

### BRIEF DESCRIPTION OF THE DRAWINGS

To easily identify the discussion of any particular element or act, the most significant digit or digits in a reference number refer to the figure number in which that element is first introduced.

FIG. 1 is a schematic diagram depicting an imaging data processing system, according to certain aspects of the present disclosure.

FIG. 2 is a schematic diagram depicting an image restoration workflow, according to certain aspects of the present disclosure.

FIG. 3 is a flowchart depicting a process for performing image restoration using a synthetic prior, according to certain aspects of the present disclosure.

FIG. 4 is a flowchart depicting a process for generating synthetic imaging data, according to certain aspects of the present disclosure.

FIG. 5 is a flowchart depicting a process for generating synthetic imaging data using frequency domain transforms, according to certain aspects of the present disclosure.

FIG. 6 is a flowchart depicting a process for improving restoration of limited-angle imaging data, according to certain aspects of the present disclosure.

FIG. 7 is a set of images depicting various sets of imaging data of sandstone rock, according to certain aspects of the present disclosure.

FIG. 8 is a set of images depicting various sets of imaging data of sandstone rock, according to certain aspects of the present disclosure.

FIG. 9 is a chart depicting the performance of various imaging restoration processes, according to certain aspects of the present disclosure.

FIG. 10 is a set of image reconstructions for a sample, according to certain aspects of the present disclosure.

FIG. 11 is a set of image reconstructions for a training volume, according to certain aspects of the present disclosure.

FIG. 12 is a set of image reconstructions for a sample, according to certain aspects of the present disclosure.

FIG. 13 is a set of image reconstructions for a sample (e.g., the sample of FIG. 12), according to certain aspects of the present disclosure.

FIG. 14 is a block diagram of an example system architecture for implementing features and processes of the present disclosure.

FIG. 15 is a schematic block diagram depicting a system for reconstructing computed tomography (CT) images from sparse-view imaging data, according to certain aspects of the present disclosure.

FIG. 16 is a flowchart depicting a process for reconstructing an image from sparse-view computed tomography (CT) imaging data, according to certain aspects of the present disclosure.

FIG. 17 is a flowchart depicting a process for creating a trained image quality model using full-view imaging data, according to certain aspects of the present disclosure.

FIG. 18 is a flowchart depicting a process for providing a residual neural network architecture to a machine learning model, according to certain aspects of the present disclosure.

FIG. 19 is a flowchart depicting a process for generating reconstructions using filtered back-projection, according to certain aspects of the present disclosure.

FIG. 20 is a flowchart depicting a process for constructing a full-view spectral noise field, according to certain aspects of the present disclosure.

FIG. 21 is a flowchart depicting a process for normalizing training inputs using computed mean and standard deviation, according to certain aspects of the present disclosure.

FIG. 22 is a flowchart depicting a process for deriving an edge mask and applying edge-weighted loss during training, according to certain aspects of the present disclosure.

FIG. 23 is a flowchart depicting a process for constructing a throughput-conditioned spectral noise field, according to certain aspects of the present disclosure.

FIG. 24 is a flowchart depicting a process for padding, rescaling, and applying a mean-squared error loss to a throughput-conditioned machine learning model, according to certain aspects of the present disclosure.

FIG. 25 depicts example reconstruction results comparing various methods for sparse-view CT imaging, according to certain aspects of the present disclosure.

### DETAILED DESCRIPTION

Certain aspects and features of the present disclosure relate to image restoration techniques making use of a synthetic prior. An imaging process, such as X-ray microscopy can generate imaging data from an original subject. The imaging data can be subject to a first imaging restoration process to generate the synthetic prior, which acts as an approximation for the original subject. This synthetic prior can be subjected to a forward imaging operator to generate synthetic imaging data. A second imaging restoration process can then be trained using the synthetic imaging data as an input and the synthetic prior as a desired output. The trained second imaging restoration process can then be applied to the original received imaging data to generate restored imaging data. The restored imaging data would thus provide a more accurate approximation for the original subject than the synthetic prior.

While described herein with reference to imaging in general, certain aspects and features of the present disclosure are especially suitable for X-ray imaging and X-ray reconstruction, and especially with respect to X-ray microscopy. X-ray reconstruction, among other imaging techniques, can be prone to noise and sampling artifacts, missing wedge artifacts, beam hardening, interior tomography artifacts, cone angle/laminography artifacts, and the like. Certain aspects and features of the present disclosure are especially suitable for transmission imaging studies (e.g., X-ray imaging studies), in which electromagnetic radiation is directed at a subject being imaged and imaging is based on electromagnetic radiation detected after passing through the subject. For example, in X-ray imaging, X-rays are directed to a sample and are absorbed or scattered by the sample as the X-rays travel through the sample. The X-rays not absorbed or scattered away are transmitted through and modulated by the sample before being received by a detector. Certain aspects and features of the present disclosure are especially suitable for tomography imaging, in which multiple acquisitions are acquired of the subject at multiple different angles to permit reconstruction of one or more slices of the subject.

An original subject can be represented by a structure function, S. The original subject can be any suitable subject for which imaging is desired. When the original subject is imaged, the resulting output is imaging data, D. Thus, imaging of the subject to obtain the imaging data can be described as performing some imaging operator, G on the structure function to create the imaging data, such as shown in equation, D=G(S).

Imaging data itself can be stored and/or manipulated in any suitable form or domain. In some cases, imaging data is raw data acquired in the projection domain, although that need not always be the case. In some cases imaging data can refer to images created from reconstruction of raw imaging data, such as volumetric reconstructions. As used herein, the term projection domain refers to data representative of the individual projections acquired of the sample themselves. Such data is generally in a 2-dimensional format (e.g., an individual radiograph), although that need not always be the case. As used herein, the term image domain refers to data that has been reconstructed from individual projections to generate an image or volume representative of the sample. Such data is generally in a 2-dimensional format (e.g., a reconstruction of a tomographic slice of the sample) or 3-dimensional format (e.g., a volumetric reconstruction of the sample), although that need not always be the case.

Imaging restoration processes can be applied to imaging data to improve the imaging data and make it a more accurate representation of the original structure function (e.g., the original structure). The improvements can include reconstruction of raw imaging data into two-dimensional or volumetric representations of a subject, although that need not always be the case. In some cases, an imaging restoration process includes taking a volumetric representation of a subject as input and outputting an improved volumetric representation of the subject, such as with reduced noise or other artefacts. According to certain aspects and features of the present disclosure, an imaging restoration process includes leveraging a trained machine learning model to perform some imaging task. Some imaging restoration processes, especially those based on machine learning models, when used alone, can introduce undesired effects, such as oversmoothing.

An ideal restoration process would involve applying a reverse imaging operator, G⁻¹ to the imaging data to recover the original structure function from the imaging data, such as shown in equation, S=G⁻¹(D). However, finding the ideal inverse operator is extremely difficult.

Various technologies exist to try and approximate this image restoration process for various specific image restoration tasks. For example, accounting for the sparse angle problem or noise has been attempted using Huber regularized iterative reconstruction or deep learning reconstruction. As another example, accounting for beam hardening artefacts has been attempted using polynomial beam hardening correction. These techniques, however, suffer from their own significant challenges. They can often be computationally complex, require hyper parameter tuning, and can often introduce their own incremental artifacts above what may be desired.

Certain aspects and features of the present disclosure leverage a first imaging restoration process, Gₐ⁻¹, to create an image (e.g., a 2D, 3D, or other image) that is used as an intermediate step to generating a final, restored image. This intermediate image, being already processed using a first imaging restoration process, has a feature distribution that approximates that of the original structure function. This intermediate image can be known as a synthetic prior, Sₐ, since it is used as a synthetic “sample” that contains a feature distribution close to the real sample, constructed using the prior information available in the sample, i.e., derived from the imaging data. The synthetic prior can be determined according to the equation, Sₐ=Gₐ⁻¹(D).

The synthetic prior can then be degraded using some forward imaging operator, G_{b}. The forward imaging operator should approximate the overall impact of the degradation process (e.g., the degradation from imaging the original subject to output the received imaging data) as faithfully as possible. In some cases, an accurate forward system model may be used. In some cases, a robust heuristic model can be used. Other techniques may be used. The result of applying the forward imaging operator to the synthetic prior is synthetic imaging data, D_{b}, which can be determined according to the equation, D_{b}=G_{b}(Sₐ).

This synthetic imaging data can then be used as the input for a second imaging restoration process, G_{c}⁻¹. As for many problems a forward problem is easier to solve to a high fidelity than an equivalent inverse problem with no apriori prior information. Since both the synthetic imaging data is known and the synthetic prior is known, the second imaging restoration process can be optimized more effectively. Knowing both the input and output for the second imaging restoration process allows optimization of any hyperparameters (α) in the second imaging restoration process to maximize the fidelity of recovery, as seen in the equation, Sₐ=G_{c}⁻¹(D_{b}, α), α=argmin((Sₐ−G_{c}⁻¹(D_{b}))²), where argmin is indicative of optimizing the hyperparameter(s), α, until the difference between the synthetic prior and the output of the second imaging restoration process applied to the synthetic imaging data is at a minimum.

In the aforementioned example, the hyperparameters a can be optimized using a L₂ norm (mean square error), but in some cases, other loss functions are used.

The inverse imaging operator G_{c}⁻¹ can be any reconstruction/restoration operation with tunable hyperparameters. Notably, convolutional neural networks (CNNs) provide a generally applicable tool that can be adapted to any image restoration task, given the presence of an input dataset (e.g., D_{b}) and a target/label dataset (e.g., Sₐ).

Training the second imaging restoration process on the synthetic imaging data derived from the synthetic prior is especially beneficial in obtaining high-quality restored imaging data. Any image restoration process trained based on the received imaging data instead of the synthetic imaging data would be optimized to remove any features and introduce any artifact introduced by the imaging restoration process, and thus would not offer any performance benefit relative to the first imaging restoration process, other than potentially a speed benefit if a more computationally efficient approach was used for the trained imaging restoration process than is available for the first imaging restoration process.

The degradation process (e.g., the forward imaging operator) should not introduce any features that would not be acceptable to remove using the trained imaging restoration process, G_{c}⁻¹. Removed features should be absent in both the synthetic imaging data and the synthetic prior, so the hyperparameters will not be optimized to remove them. Further, any artifacts that are introduced when using the first imaging restoration process, Gₐ⁻¹, should be present in both the synthetic prior and the synthetic imaging data. This correlation should lead the trained imaging restoration process, G_{c}⁻¹, to not be trained to introduce these artifacts as they are correlated between both the input and the labels (e.g., the desired output, which is the synthetic prior).

Once the second imaging restoration process has been trained (e.g., optimized), it can be applied to the original imaging data to generate the restored imaging data (S_{b}), such as seen in the equation, S_{b}=G_{c}⁻¹(D).

While described herein with respect to one iteration of received imaging data, synthetic prior, and synthetic imaging data, in some cases additional iterations can be used. In such cases, an additional iteration can use the restored imaging data from a prior iteration as the synthetic prior for the additional iteration. For example, an original structure function, S, can be used to generate imaging data that is then used to generate a synthetic prior, Sₐ, which can be used to generate synthetic imaging data D_{b}. When a single iteration is used, a second imaging restoration process can be trained using D_{b} and Sₐ, and that second imaging restoration process can be used to generate the restored imaging data, S_{b} from the original imaging data. However, if a second iteration is used, the restored imaging data, S_{b} can function as the synthetic prior used to generate additional synthetic imaging data, D_{c}, which can be used with S_{b} to train a third imaging restoration process, which can then be used to generate further restored imaging data (e.g., by applying the third imaging restoration process to the original imaging data). Any number of iterations could be used.

This overall workflow can be applied across a wide range of image restoration tasks, by using a first imaging restoration process, Gₐ⁻¹ to initially construct the synthetic prior and a forward imaging operator, G_{b}, to construct the synthetic imaging data. Apart from these operators, the second imaging restoration process, G_{c}⁻¹, can be re-used, retraining the network to the next imaging restoration task.

In a first example use case, certain aspects and features of the present disclosure can be used to remove undesired noise and artifacts from initial imaging data. An example first imaging restoration process can be used to try and improve initial imaging data by subsampling projection data into two subsets (A and B), such as those representing even and odd projections, respectively. These two subsets of data are reconstructed into two volumes and a symmetrical noise-to-noise model (with loss computed from the mapping of A to B and B to A together) is trained to map from one to the other having the effect of both denoising and removing sparse sampling artifacts at the same time. (See e.g., U.S. Patent Publication No. US2003/0009951A1, the disclosure of which is incorporated by reference herein). In such a case, a final estimate of the structure function is given by passing a reconstruction created using all projections or a subset of projections through the trained network. The result of this first imaging restoration process is normally intended to be a final output (e.g., to be displayed to a user and/or used for further analysis).

Such a first imaging restoration process works well, but has some drawbacks. First, the network is trained with an input noise distribution found from half the total projection number, which is different from the noise distribution of the data used for final inference. Using just a projection subset for inference was found experimentally to yield less accurate results than when using the full projection number. This difference in noise distribution means that the network will tend to over-smooth the data relative to some theoretical optimal denoiser. Second, because a noise-to-noise approach is used, any imaging artifact that is correlated between the subsets will be retained in the final image. Spurious image signal in X-ray reconstruction is a combination of sparse sampling artifact and true noise. The noise will be uncorrelated but sparse sampling artifacts often have a degree of correlation between the projection subsets. The angular offset between the projection subsets and the fact that the network is trained in a symmetric fashion minimizes the impact of this suboptimal denoising, however it may still be noticeable in the final reconstructed volume.

According to certain aspects and features of the present disclosure, this first imaging restoration process can be used to generate the synthetic prior. Generally, this synthetic prior will be used as an intermediate and not provided as any sort of final output (e.g., not displayed to a user or used for substantive analysis), although that need not always be the case. Since the synthetic prior need not be displayed to a user or otherwise relied upon for substantive analysis, the first imaging restoration process used to generate the synthetic prior can be adjusted to produce intermediate imaging data that is more useful for the generation of synthetic imaging data and training of a second imaging restoration process, at the cost of visual or other degradations that may make the synthetic prior otherwise unsuitable for a final output.

Next, a forward imaging operator (e.g., forward model) can be used to re-introduce the noise and sampling artefacts to the level of the input dataset. While one could use an accurate system forward model (e.g., forward projecting the synthetic prior, simulating noise, adding it to the projection, and then re-back projecting), such an approach is problematic for several reasons.

First, the problem is only well determined for samples that are wholly contained inside the Field of View. If the same extends beyond the field of view (e.g., an interior tomography), then the reconstructed volume does not constitute full information about the sample and its forward projection will not properly correspond to the data. Compensating for this effect is imperfect and corresponds to an inaccurate estimation of noise. Second, the forward and backward projection operation involves multiple interpolation steps that introduce incremental image blur. Finally, such a forward model may induce correlations between the spurious image signal and the structure function, leading to suboptimal trained removal.

However, according to certain aspects and features of the present disclosure, a heuristic model can be used. First, the imaging operator is approximated as a simple addition of noise, such as according to the equation, D_{b}=G_{b} (Sₐ)~Sₐ+N, where D_{b} is the synthetic imaging data, G_{b} is the forward imaging operator, Sₐ is the synthetic prior, and N is a noise function, here represented in the volumetric domain.

Noise in X-ray imaging is heteroscedastic, spatially variable, and spatially anisotropic. This is theoretically tractable in the projection (e.g., transmission) domain as approximately Poissonian, however its representation in the volume domain depends on structures elsewhere in the object and is extremely hard to predict based on the values of the synthetic prior alone. However, an approximation for the initial noise distribution (Nᵢ) with a full description of the second order complexities can be calculated as the residual from the data of the synthetic prior, such as according to equation Nᵢ=D−Sₐ. This spurious signal field cannot be used to recover an image D_{b}, however it can be analyzed in detail to give insight into the specific complexities of the noise field for this dataset.

First, the noise field can be analyzed to estimate a local spatially variant standard deviation, σᵢ, such as according to the equation:

$${σ}_{i} = \sqrt{{\overline{{N}_{i}^{2}}}^{3 x 3 x 3}} ,$$

where the local average of the square error (the object

$${\overline{{N}_{i}^{2}}}^{3 x 3 x 3} ,$$

taken here over a small 3-dimensional 3×3×3 window, although other suitable window sizes can be used) is a representation of the local variance, as Sa can be viewed as an estimate of local measurement mean. This spatially variant standard deviation can then be used to seed a heteroscedastic normal random generator to generate a new, random noise field, G (e.g., random gaussian field).

This new noise field, however, remains an incomplete model of volumetric X-ray noise, as while it accounts for the spatial variability and heteroscedasticity of the noise field, it remains spatially isotropic and white. In other words, it has no local spatial auto-correlation in any dimension. To account for this incompleteness, the frequency domain (e.g., Fourier spectrum) of Nᵢ can be considered. A Fourier spectrum of an image is a complex representation of the spatial frequencies in the image, and can be represented in many ways, however a particularly informative one is to split up the complex transform into two components: magnitude and phase. Given a complex number, C, where C=a+bi, the magnitude can be represented by mag(C)=\sqrt{a²+b²} and the phase can be represented by phase(C)=arctab(b/a).

The magnitude is a representation of the spatial frequency distribution in the image and the phase is a representation of the specific position of that frequency distribution. To say that X-ray noise is non-white is to say it has a non-uniform distribution of Fourier magnitudes, and to say it is anisotropic is to say that it is different in the 3 dimensions of the multidimensional Fourier transform.

Matching a noise field to the initial noise distribution can be achieved by matching the Fourier magnitudes of the noise field and the initial noise distribution, but not the phases. As the volumetric expression of spurious signal in X-ray imaging is spatially variable, only matching of Fourier magnitudes should be enforced between the initial noise distribution and the random gaussian field. This matching can be done by locally chunking the volume (e.g., into small cubes of geometry 64×64×64 voxels). The complex Fourier domain representation of the new noise field can be generated by taking the magnitudes from the initial noise distribution (e.g., N_{imag}=mag(fft(Nᵢ))) and combining it with the phase from the random gaussian field G (e.g., Gₚₕₐₛₑ=phase(fft(G))), such as seen in the following equation:

$$fft ( N ) = {N}_{i mag} \times cos ( {G}_{p h a s e} ) + {N}_{i mag} \times sin ( {G}_{p h a s e} ) i .$$

The final synthetic noise field (N) is then just the real component of the inverse Fourier transform of this object, as seen in the following equation:

$$N = real ( ifft ( {N}_{i mag} \times cos ( {G}_{p h a s e} ) + {N}_{i mag} \times sin ( {G}_{p h a s e} ) i ) ) ,$$

where fft(x) and ifft(x) represent the n-dimensional Fourier transform and n-dimensional inverse Fourier transform, respectively, real(x) is a real component function (e.g., function to extract the real component), and i is \sqrt{−1}. This approach can be applied, chunk by chunk, across the entire image Nᵢ and G to create a new noise field N (e.g., the synthetic noise field) having equivalent local Fourier magnitudes to Nᵢ but fully decorrelated phase.

In some alternate implementations, the phase information could be created with a simply uniform random number generator rather than by taking the Fourier transform of the random field G.

Once the synthetic noise field is obtained, it can be applied to the synthetic prior to generate synthetic imaging data. A neural network can then be trained to map the synthetic imaging data to the synthetic prior, with this network being the optimized inverse operator G_{c}⁻¹. This network can then be applied to the original imaging data to output the restored imaging data, S_{b}, (e.g., a close approximation of the original structure function, S).

In a second example use case, certain aspects and features of the present disclosure can be used to improve reconstructions where the projection dataset is missing certain angles. Typically X-ray tomographic projections are taken through a sequence of angles evenly spaced through either 180+fan angle degrees (e.g., for cone beam CT imaging) or 360 degrees, yielding a good reconstruction. Sometimes experimental constraints limit the number of angles available to reconstruction, particularly when using in situ equipment that requires highly attenuating/X-ray opaque equipment oriented such that it is not possible to acquire certain views. Traditional reconstruction yields artifacts in the form of streaks and anomalous reconstructed attenuations, causing ambiguity in analysis and issues with interpretation.

Unlike the noise and sparse sampling artifacts described in the first example use case, there is no existing process of record to correct for these artifacts (Gₐ⁻¹). Certain aspects and features of the present disclosure relate to a new process for correcting for these artifacts.

First the dataset was reconstructed using the missing wedge projection data, both using all projections and offset projection subsets. A noise-to-noise model was trained and inferred to create a denoised volume which was then subtracted from the full projection reconstruction to give an estimate of the noise field (N).

Then a volume of arbitrary objects was created and forward projected into both the angle range available for missing wedge reconstruction and a full 360 degree series. Forward projecting the volume of arbitrary objects can include determining projective geometry associated with the received imaging data and forward projecting using that same projective geometry (e.g., source distance, detector distance, dither corrections, etc.). The two projection datasets were then re-back projected into the volume domain to create a pair of images—one corresponding to the missing wedge dataset, one for the full projection series.

The datasets were then scaled such that the mean and standard deviation of the synthetic data reconstructions matched that of the of the real data reconstructions, and the noise field was added to the missing wedge reconstruction. A residual network was trained to map from the missing wedge (+ noise) synthetic data reconstruction to the full angle synthetic data reconstruction, creating a network that has the effect of both removing the missing angle artefacts and the data noise, and this network was then applied to the real data missing wedge reconstruction (e.g., the received imaging data).

The resultant dataset constitutes the synthetic prior (Sₐ). However, this synthetic prior may still exhibit significant residual artifacts due to the imperfect correlation between the feature distribution in the synthetic object image and the feature distribution in the imaged structure.

A forward model (G_{b}) can be used to construct a synthetic data representation (D_{b}) from the synthetic prior. In this example case, as the restoration is related to missing projection domain information, first the synthetic prior is forward projected, creating a new projection dataset through a projective geometry corresponding to the original data. For a full field of view (FFOV) tomography the full information is known about the sample, but for interior tomography, correction is needed for the attenuation coming from outside the field of view to obtain an accurate forward projection model. This can be achieved by subtracting the forward projection (FP) from the data (D) to create a representation of the attenuation corresponding to materials outside the field of view (O) according to the equation, O=D−FP(Sₐ).

This representation, however, will also represent all the spurious signal in D, as the forward projection does not contain this information, so this representation can be further denoised (e.g., denoised around a local kernel). This denoising could be a local mean, or gaussian filter, or similar simple denoising function, to create an outside of field of view representation O_{f} according to the equation,

$${O}_{f} = {\overline{O}}^{3 x3} .$$

As this representation is only used to correct the attenuation values used for back projection, and the features in it are poorly correlated across views, the application of a spatial blur would not modify the reconstructed volume. It is therefore re-added to the forward projection set to create a final simulated projection set. This is then back projected (BP), creating a new dataset that has simulated missing wedge artifacts in it. Finally, the pre-estimated noise field (N) is added back to the missing wedge, creating a high fidelity synthetic data representation (D_{b}). The construction of D_{b} is therefore represented by the equation:

$${D}_{b} = BP ( FP ( {S}_{a} ) + {( D - FP ( {S}_{a} ) )}^{3 x 3} ) + N .$$

The noise field (here described as the original noise field) can be re-synthesized with the spectral matched noise method described above. A final restoration network can then be trained regressing this D_{b} representation against the synthetic prior Sₐ described above. This can then be applied to the original data D to create the restored imaging data (S_{b}).

Certain aspects and features of the present disclosure improve a computing device's ability to process imaging data, especially with respect to imaging data from imagers like X-ray microscopes and the like. Certain aspects and features of the present disclosure improve the technical field of image processing by enabling new techniques for further improving image restoration. Certain aspects and features of the present disclosure improve the technical field of X-ray microscopy (and similar fields) by improving imaging restoration abilities, which can result in the need to collect fewer projections to achieve a desired image quality, and thus reduce imaging time and required data storage. Various other improvements are also achieved.

These illustrative examples are given to introduce the reader to the general subject matter discussed here and are not intended to limit the scope of the disclosed concepts. The following sections describe various additional features and examples with reference to the drawings in which like numerals indicate like elements, and directional descriptions are used to describe the illustrative embodiments but, like the illustrative embodiments, should not be used to limit the present disclosure. The elements included in the illustrations herein may not be drawn to scale.

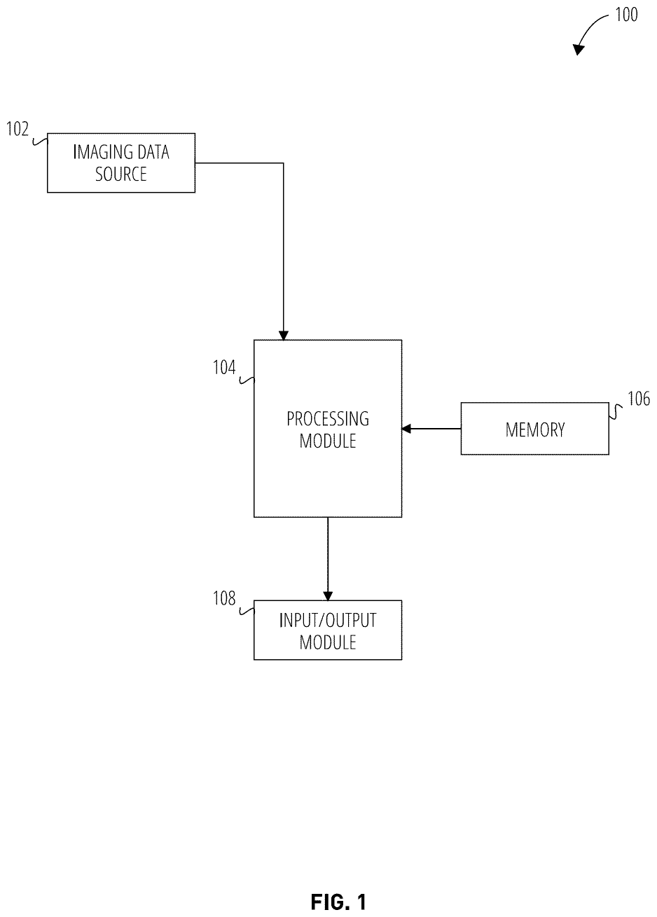

FIG. 1 is a schematic diagram depicting an imaging data processing system 100, according to certain aspects of the present disclosure. The imaging data processing system 100 (e.g., control system) can include an imaging data source 102 that provides imaging data to a processing module 104. The imaging data source 102 can be any suitable source of imaging data, such as an imager (e.g., an imaging machine, such as an X-ray microscope or a CT scanner), a database of imaging data, a local memory storing imaging data, a removable memory storing imaging data, or the like. Certain aspects and features of the present disclosure are especially useful when the imaging data source 102 is an imager, such as an X-ray microscope.

The processing module 104 can process imaging data from the imaging data source 102. In some cases, the processing module 104 can control the imaging data source 102.

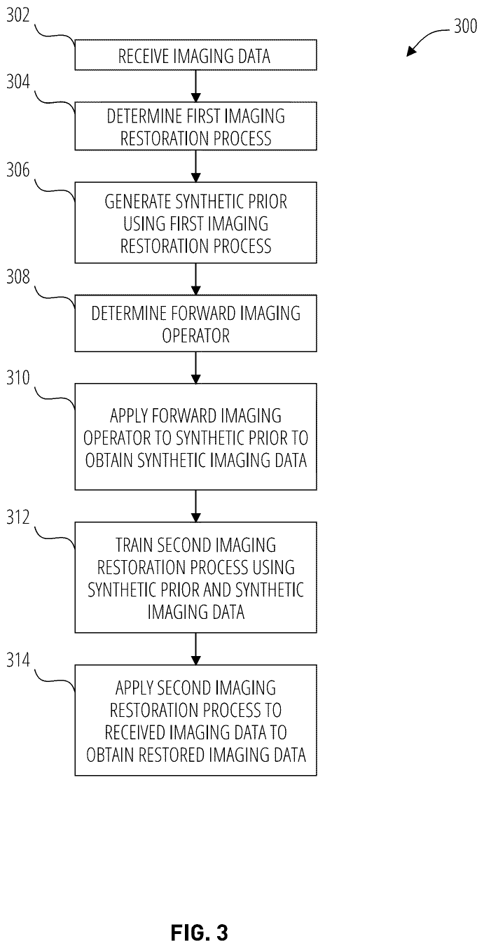

Processing module 104 can perform various operations on received imaging data, such as those described here (e.g., process 300 of FIG. 3). Memory 106 can store machine-readable instructions that enable the processing module 104 to perform its various operations. Memory 106 can also store untrained or trained models (e.g., machine learning algorithms or neural networks) that can be used in the restoration of imaging data. Memory 106 can store received imaging data, any intermediate data (e.g., synthetic prior data), and any output data (e.g., restored imaging data). Memory 106 can be implemented as locally accessible memory and/or remotely accessible memory (e.g., accessible via a network such as a local area network, a wide area network, a cloud network, or the Internet).

The processing module 104 can use imaging data (e.g., received imaging data and/or synthetic imaging data) to train one or more neural networks, such as an artificial neural network (ANN) (e.g., a deep neural network (DNN), a convolutional neural network (CNN), or the like), and/or use such a trained neural network to process imaging data or a reconstructed volume into improved imaging data or an improved reconstructed volume. For example, received imaging data can be processed into a synthetic prior using a first imaging restoration process (e.g., a first, trained neural network), synthetic imaging data can be processed into a recreation of the synthetic prior to train a second imaging restoration process (e.g., a second neural network), and the received imaging data can be processed into restored imaging data using the trained, second imaging restoration process (e.g., the trained second neural network). In some cases, training of a first neural network can be used to facilitate training of a second neural network, such as through transfer learning.

The processing module 104 can also carry out reconstruction of imaging data (e.g., raw imaging data or improved imaging data) to generate reconstructed volumes (e.g., converting a set of acquired projections into a three-dimensional reconstructed volume). In some cases, the processing module 104 can access a pre-trained neural network from a memory 106, which can be applied as-is or can be further trained. In some cases, the pre-trained neural network can be a neural network that is generated using a federated learning technique, in which multiple trained neural networks can be collected and combined to generate a collaborative neural network that is distributed as the pre-trained neural network. In such cases, each pre-trained neural network can be associated with the same category of sample and/or the same or similar acquisition parameters, and the pre-trained neural network accessed by the processing module 104 can be accessed based on a provided category and/or set of acquisition parameters.

An input/output module 108 can be coupled to the processing module 104 to receive user input and provide output to a user. Any suitable input/output devices can be implemented in the input/output module 108, such as a keyboard, a mouse, a display (e.g., computer monitor), a touchscreen, light emitting diodes (LEDs) or other light sources, buttons, and the like. The processing module 104 can present reconstructions (e.g., reconstructed volumes) and/or further information derived therefrom to a user via the input/output module 108. In some cases, the input/output module 108 can store imaging data (e.g., received imaging data and/or restored imaging data), a reconstructed volume, and/or a trained neural network (e.g., on a local memory, removable memory, or network-accessible memory). In some cases, a neural network trained using a processing module 104 can be stored in association with the imaging data and/or reconstructed volume.

In some cases, any of the imaging data source 102, the processing module 104, the memory 106, and the input/output module 108 can be incorporated into one or more housings in any suitable combination. Any combination of one or more of the imaging data source 102, the processing module 104, the memory 106, and the input/output module 108 can be implemented locally (e.g., on the same device as one another or on devices coupled by a bus or local area network) or remotely (e.g., via a wide area network, the Internet, or a cloud network). In an example, a processing module 104 can be implemented on a user's laptop computer, the imaging data source 102 can be implemented on a cloud-based health record database (e.g., one or more servers accessible via the Internet), and at least a portion of the memory 106 can be implemented on a separate cloud-based analysis database (e.g., one or more servers accessible via the Internet).

In another example, the processing module 104 can be incorporated into an imaging data source 102, such as a computer for processing imaging data that is also used to control an X-ray microscope. In another example, the processing module 104 can be incorporated into an individual computer that accesses, via a network-accessible database, imaging data supplied from a separate CT scanner or X-ray microscope.

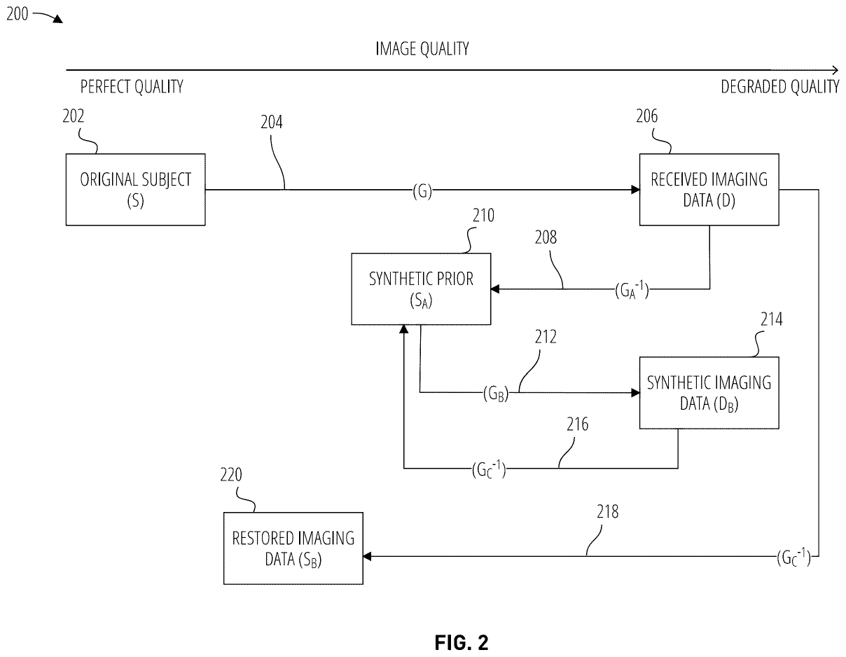

FIG. 2 is a schematic diagram depicting an image restoration workflow 200, according to certain aspects of the present disclosure. The workflow 200 can be implemented using any suitable system, such as imaging data processing system 100 of FIG. 1. The workflow 200 depicts various imaging operators that connect various elements and imaging data. The workflow 200 is arranged on an image quality scale from left to right, with the leftmost end of the scale representing perfect quality, which by definition is the original subject original subject 202 being imaged.

The original subject 202 can be represented by a structure function S. When the original subject 202 is imaged (e.g., using an X-ray microscope), the result is received imaging data 206 (D). Various aspects of the imaging process (e.g., settings, mechanical limitations, etc.) result in the received imaging data 206 being degraded by some amount. This degradation can be represented by imaging device function 204, G, such that D=G(S).

The received imaging data 206 can be used to generate a synthetic prior 210 (Sₐ) through a first imaging restoration process 208 (G_{A}⁻¹), such that Sₐ=G_{A}⁻¹(D).

The synthetic prior 210 can then be used to generate synthetic imaging data 214 (D_{b}) by applying a forward imaging operator 212 (G_{b}), such that D_{b}=G_{b}(Sₐ).

The synthetic imaging data 214 can then be supplied as input to a second imaging restoration process 216 (G_{c}⁻¹) to generate recreations of the synthetic prior 210. By using the synthetic prior 210 as a desired output (e.g., as “labeled data”), the second imaging restoration process 216 can be trained/optimized until the second imaging restoration process 216 is able to generate a sufficiently suitable recreation of the synthetic prior 210 from the synthetic imaging data 214.

Then, the same second imaging restoration process 218 can be applied to the received imaging data 206 to generate the restored imaging data 220 (S_{b}), such that S_{b}=G_{c}⁻¹(D). The restored imaging data 220 is the improved imaging data that is improved over the received imaging data 206 and the synthetic prior 210.

The depiction of the received imaging data 206, synthetic prior 210, synthetic imaging data 214, and restored imaging data 220 at certain locations along an image quality scale is for illustrative purposes. In some cases, one or more of these elements may be located at different locations along the image quality scale. For example, the synthetic imaging data 214 may be located to the left or right of the received imaging data 206, rather than at the same point along the scale.

FIG. 3 is a flowchart depicting a process 300 for performing image restoration using a synthetic prior, according to certain aspects of the present disclosure. Process 300 can be performed using any suitable hardware, such as imaging data processing system 100 of FIG. 1.

At block 302, imaging data is received. Imaging data can be received from any suitable source, such as directly from an imager (e.g., an X-ray microscope), from a database of previously acquired imaging data, from a removable storage device, or the like. The imaging data can be in any suitable form. In some cases, the imaging data is raw imaging data representative of a set of projections acquired of a subject. Such raw imaging data may have undergone some degree of preprocessing prior to being received at block 302, although that need not always be the case. In some cases, the imaging data is an image reconstruction based on a set of projections acquired of a subject. In some cases, receiving imaging data at block 302 includes receiving metadata associated with the imaging data. In some cases, receiving imaging data at block 302 includes operating (e.g., sending signals to operate) an imager to acquire the imaging data.

At block 304, a first imaging restoration process can be determined. The first imaging restoration process can be determined, optionally based at least in part on the received imaging data. In some cases, a pre-determined first imaging restoration process can be used. In some cases, a pre-determined first imaging restoration process can be selected from a set of available pre-determined first imaging restoration processes, such as based on the received imaging data and/or metadata associated with the received imaging data (e.g., a particular first imaging restoration process can be selected for a particular imager and/or imaging settings used during acquisition of the received imaging data).

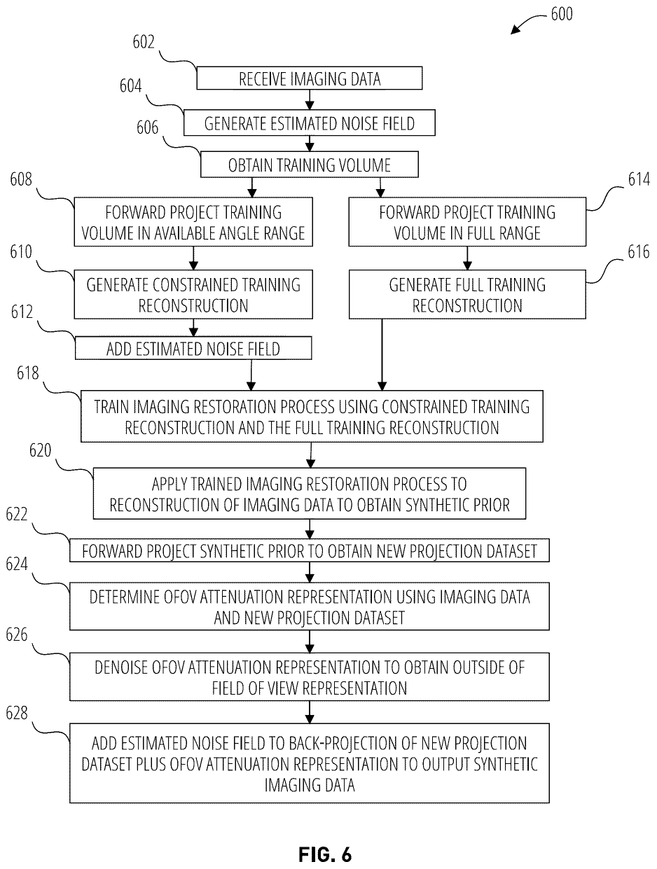

At block 306, a synthetic prior is generated using a first imaging restoration process. The first imaging restoration process can be any suitable image restoration process (e.g., an inverse imaging operator) that attempts to approximate a representation of the original subject that was imaged. Generally, the first imaging restoration process can be a state-of-the-art imaging restoration process, such as a state-of-the-art deep-learning-based image restoration/reconstruction tool (e.g., ZEISS® DeepRecon Pro™). In some cases, the first imaging restoration process can include other techniques, such as those described with reference to process 600 of FIG. 6.

The synthetic prior can be a form of imaging data. Often, the received imaging data will be in the form of a dataset of projections (e.g., in the projection domain) while the synthetic prior is in the form of a reconstruction (e.g., in the image domain or volume domain) based on that dataset of projections, although that need not always be the case.

At block 308, a forward imaging operator is determined. The forward imaging operator can be determined, optionally based at least in part on the synthetic prior and the received imaging data. In some cases, a pre-determined forward imaging operator can be used. In some cases, a pre-determined forward imaging operator can be selected from a set of available pre-determined forward imaging operators, such as based on the synthetic prior, the received imaging data, and/or metadata associated with the received imaging data (e.g., a particular forward imaging operator can be selected for a particular imager and/or imaging settings used during acquisition of the received imaging data).

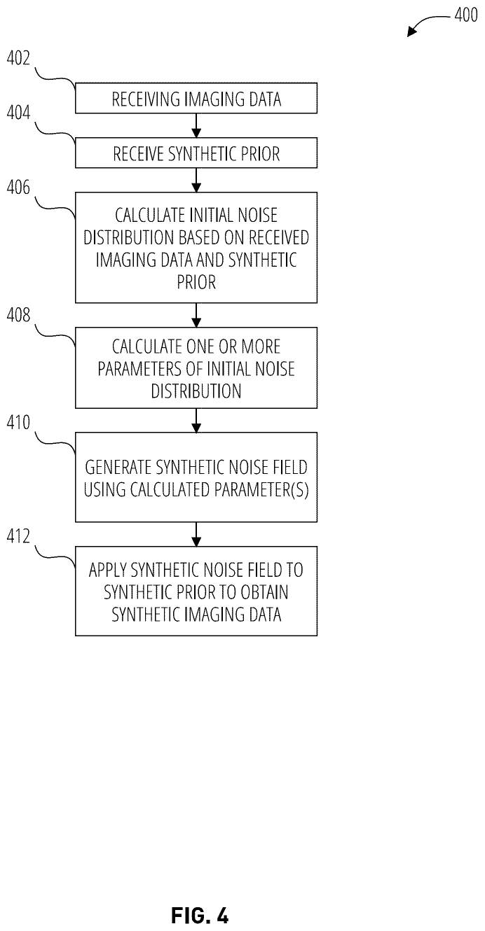

In some cases, however, determining the forward imaging operator can include extracting certain information from the synthetic prior and/or the received imaging data. For example, in some cases, determining the forward imaging operator at block 308 can include extracting information about the noise distribution of the received imaging data, such as described with reference to process 400 of FIG. 4.

At block 310, the forward imaging operator is applied to the synthetic prior to generate synthetic imaging data. The synthetic imaging data can be a form of imaging data. The synthetic imaging data can be in the same form or domain as the received imaging data. For example, in some cases, the received imaging data from block 302 is in the projection domain, the synthetic prior of block 306 is in the image domain, and the synthetic imaging data of block 310 is in the projection domain. The synthetic imaging data can be a set of projections that represent the data an imager would output if it had acquired imaging data from the synthetic prior. The synthetic imaging data is referred to as synthetic because it is generated from the synthetic prior. Applying the forward imaging operator at block 310 can include applying the forward imaging operator such that the synthetic imaging data has the same level of noise and/or sampling artifacts as the received imaging data.

At block 312, a second imaging restoration process is trained using the synthetic prior and the synthetic imaging data. Training the second imaging restoration process can include using the synthetic imaging data as an input and using the synthetic prior as a desired output. Thus, as adjustments are made to the second imaging restoration process (e.g., adjustments to weights in nodes of a neural network) during training, the output of the second imaging restoration process supplied with the synthetic imaging data can be compared with the synthetic prior. Training the second imaging restoration process can include minimizing a loss function that is based on a difference between the synthetic prior and the output of the second imaging restoration process supplied with the synthetic imaging data. Any suitable machine learning techniques can be used to train the second imaging restoration process. The second imaging restoration process can take any suitable form, although often includes a neural network.

At block 314, the trained second imaging restoration process can be applied to the received imaging data to output the restored imaging data. The restored imaging data is imaging data that has been improved through the synthetic prior restoration process. The restored imaging data can be in any suitable form. The restored imaging data can be in the same form or domain as the synthetic prior.

While described with certain blocks in a certain order, the process 300, the sequence may be altered without departing from the scope of the present disclosure. For example, some blocks may be performed in parallel or in a different sequence that does not materially affect the function of the process 300. In other examples, different components of an example device or system that implements the process 300 may perform functions at substantially the same time or in a specific sequence. In some cases, process 300 may include fewer blocks or additional blocks. For example, in some cases process 300 may include an additional block after block 314 related to outputting the restored imaging data, such as via displaying the restored imaging data using a display device.

FIG. 4 is a flowchart depicting a process 400 for generating synthetic imaging data, according to certain aspects of the present disclosure. Process 400 can be performed using any suitable hardware, such as imaging data processing system 100 of FIG. 1.

At block 402, imaging data is received. Receiving imaging data at block 402 can be the same as or similar to block 302 of FIG. 3.

At block 404, a synthetic prior is received. Receiving the synthetic prior at block 404 can include generating the synthetic prior (e.g., as described herein and with reference to block 306 of FIG. 3). In some cases, receiving the synthetic prior can include accessing a previously generated synthetic prior from a memory.

At block 406, an initial noise distribution is calculated based on the received imaging data from block 402 and the synthetic prior from block 404. The initial noise distribution is a noise field that is imparted on the received imaging data from the imaging process. The initial noise distribution can be calculated as the residual remaining after subtracting the synthetic prior from the received imaging data (e.g., subtracting an image domain representation of the received imaging data from an image domain representation of the synthetic prior). This residual, which includes the noise removed during the first imaging restoration process, can be used as an estimation or representation of the initial noise distribution.

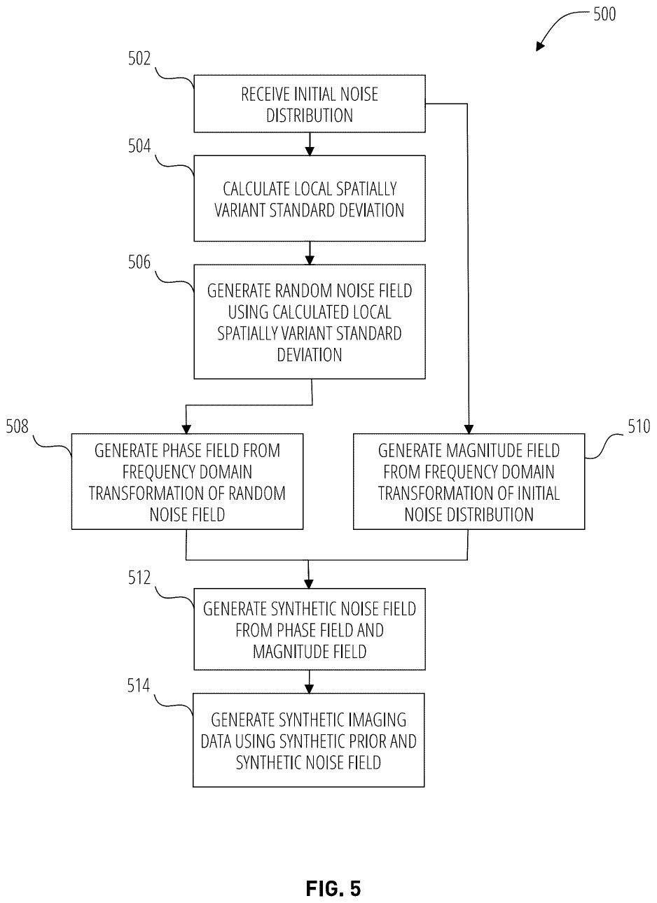

At block 408, one or more parameters of the initial noise distribution are calculated. Calculating the one or more parameters of the initial noise distribution can include determining any suitable parameter of the initial noise distribution from the estimation of block 406. These parameters can include any measurable metric of the initial noise distribution, such as a local spatially variant standard deviation (e.g., as described herein and with respect to block 504 of FIG. 5). In some cases, calculating the one or more parameters at block 408 includes calculating a magnitude distribution of a Fourier transform of the initial noise distribution (e.g., as described herein and with respect to block 510 of FIG. 5).

At block 410, a synthetic noise field is generated based at least in part on the one or more parameters from block 408. The synthetic noise field can be generated to have one or more parameters that match or substantially match (e.g., within 1%, 2%, 3%, 4%, 5%, 6%, 7%, 8%, 9%, or 10% of) those of the initial noise distribution. In some cases, generating the synthetic noise field can occur as described herein or with reference to process 500 of FIG. 5.

At block 412, the synthetic noise field can be applied to the synthetic prior to obtain synthetic imaging data. Applying the synthetic noise field to the synthetic prior can include adding the synthetic noise field to the synthetic prior as at least part of the forward imaging operator.

While described with certain blocks in a certain order, the process 400, the sequence may be altered without departing from the scope of the present disclosure. For example, some blocks may be performed in parallel or in a different sequence that does not materially affect the function of the process 400. In other examples, different components of an example device or system that implements the process 400 may perform functions at substantially the same time or in a specific sequence. In some cases, process 400 may include fewer blocks or additional blocks.

FIG. 5 is a flowchart depicting a process 500 for generating synthetic imaging data using frequency domain transforms, according to certain aspects of the present disclosure. Process 500 can be performed using any suitable hardware, such as imaging data processing system 100 of FIG. 1.

At block 502, an initial noise distribution can be received. The initial noise distribution can be received from memory or can be calculated (e.g., as described herein and with reference to block 406 of FIG. 4).

At block 504, a local spatially variant standard deviation can be calculated from the initial noise distribution of block 502. Calculating the local spatially variant standard deviation can include determining a working window size defining the size of a local area (e.g., a 3 voxel×3 voxel×3 voxel volumetric window), then calculating the spatially variant standard deviation within that window. In some cases, block 504 includes calculating the local spatially variant standard deviation for one, some, or all voxels in the initial noise distribution. For example, a local spatially variant standard deviation field can be calculated for the entire initial noise distribution by calculating the local spatially variant standard deviation at each voxel in the initial noise distribution.

At block 506, a random noise field can be generated using the local spatially variant standard deviation calculation(s). Generating the random noise field can include generating a random noise field that has local spatially variant standard deviation(s) that match or substantially match (e.g., within 1%, 2%, 3%, 4%, 5%, 6%, 7%, 8%, 9%, or 10% of) the local spatially variant standard deviation(s) of block 504. In some cases, the random noise field can be generated to have its own local spatially variant standard deviation field that matches or substantially matches the local spatially variant standard deviation field calculated at block 504. As used herein, the term random can include truly random or pseudorandom.

At block 508, a phase field is generated from the frequency domain transformation of the random noise field from block 506. The random noise field can be passed through a frequency domain transformation (e.g., a Fourier transform) to create a frequency domain representation of the random noise field containing both phase and magnitude information at each voxel. The phase field can be generated by taking only the phase information from this frequency domain representation of the random noise field.

At block 510, a magnitude field can be generated from the frequency domain representation of the initial noise distribution. The initial noise distribution can be passed through a frequency domain transformation (e.g., a Fourier transform) to create a frequency domain representation of the initial noise distribution containing both phase and magnitude information at each voxel. The magnitude field can be generated by taking only the magnitude information from this frequency domain representation of the initial noise distribution.

At block 512, a synthetic noise field can be generated from the phase field of block 508 and the magnitude field of block 512. A complex frequency domain representation of the synthetic noise field can be generated by the equation fft(N)=N_{imag}×cos(Gₚₕₐₛₑ)+N_{imag}×sin(Gₚₕₐₛₑ)i, where N is the synthetic noise field, N_{imag} is the magnitude field, Gₚₕₐₛₑ is the phase field, and i is \sqrt{−1}. An inverse frequency domain transformation (e.g., inverse Fourier transform) can be applied to the frequency domain representation of the synthetic noise field to generate a complex representation of the synthetic noise field. The actual synthetic noise field to be used can be extracted as the real portion of the resultant complex representation of the synthetic noise field.

At block 514, synthetic imaging data is generated using the synthetic prior and the synthetic noise field. Generating the synthetic imaging data can include applying the synthetic noise field to the synthetic prior, such as described herein and with reference to block 412 of FIG. 4.

While described with certain blocks in a certain order, the process 500, the sequence may be altered without departing from the scope of the present disclosure. For example, some blocks may be performed in parallel or in a different sequence that does not materially affect the function of the process 500. In other examples, different components of an example device or system that implements the process 500 may perform functions at substantially the same time or in a specific sequence. In some cases, process 500 may include fewer blocks or additional blocks. For example, in some cases blocks 504, 506, 508 can be replaced by a block that includes generating a phase field using a uniform random number generator, in which case generating the synthetic noise field at block 512 includes using that phase field instead of one generated based on the local spatially variant standard deviation calculations.

FIG. 6 is a flowchart depicting a process 600 for improving restoration of limited-angle imaging data, according to certain aspects of the present disclosure. Process 600 can be performed using any suitable hardware, such as imaging data processing system 100 of FIG. 1.

At block 602, imaging data is received. Receiving imaging data at block 602 can be the same as or similar to block 302 of FIG. 3. The imaging data can include raw imaging data representing projections acquired through a limited set of angles, rather than a full set of angles. This limited set of angles can be referred to as the available angle range. A full set of angles may include projections from 0 degrees up through 180 degrees, 360 degrees, and/or some value between 180 degrees and 360 degrees. A limited set of angles would have one or more “wedges” of angles missing from the projection dataset. As an example, received imaging data may include projections only from 0 degrees up to 150 degrees, or from 0 degrees up to 120 degrees and 150 degrees up to 180 degrees (missing a wedge of projections between 120 degrees to 150 degrees). If the imaging data received at block 602 is not in the image domain, a reconstruction of the imaging data can be generated. Thus, the imaging data output from block 602 can be in the image domain.

At block 604, an estimated noise field is determined from the received imaging data. This estimated noise field can be determined through any suitable technique. In some cases, determining the estimated noise field can include first processing the received imaging data using a first imaging restoration process that denoises the received imaging data. The resultant denoised imaging data can be subtracted from the received imaging data to generate the estimated noise field.

At block 606, a training volume is obtained. Obtaining a training volume can include using a pre-determined training volume, selecting a pre-determined training volume from a set of available training volumes, or generating a training volume. In some cases, selecting the pre-determined training volume can include selecting the pre-determined training volume based on the received imaging data or metadata associated with the received imaging data (e.g., an indication of the type of material being examined by an X-ray microscope). In some cases, generating the pre-determined training volume can include generating a volume of arbitrary objects to simulate the scale of features present in the sample imaged to generate the received imaging data.

At block 608, the training volume from block 606 can be forward projected in the available angle range to generate a dataset of projections, for the training volume, associated with the available angle range. Any suitable forward imaging operator can be used, such as those described herein. The resultant dataset of projections can be referred to as the constrained training volume imaging data.

At block 610, a constrained training reconstruction can be generated from the constrained training volume imaging data. This constrained training reconstruction will be a reconstruction of the training volume as if the training volume were imaged only through the available angle range.

At block 612, the estimated noise field from block 604 is added to the constrained training reconstruction. As a result, the constrained training reconstruction will now exhibit a noise field that is equivalent to that of the received imaging data.

At block 614, the training volume from block 606 can be forward projected in the full range to generate a dataset of projections, for the training volume, associated with a full range of angles (e.g., 360 degrees). Any suitable forward imaging operator can be used, such as those described herein. The resultant dataset of projections can be referred to as the full training volume imaging data.

At block 616, a full training reconstruction can be generated from the full training volume imaging data. This full training reconstruction will be a reconstruction of the training volume as if the training volume were imaged through the full range of angles.

At block 618, an imaging restoration process is trained using the constrained training reconstruction and the full training reconstruction. The image imaging restoration process can take the constrained training reconstruction as input and can use the full training reconstruction as a desired output. Thus, the imaging restoration process will be trained specifically to take as input a reconstruction that is constrained to the specific limited set of angles available in the received imaging data (e.g., the available angle range) and that contains the same noise field as the received imaging data, and to output a reconstruction that is based on the full range of angles and without noise.

At block 620, the trained imaging restoration process from block 618 is applied to the imaging data from block 602 (e.g., a reconstruction of raw imaging data) to generate an improved reconstruction. In some cases, this improved reconstruction can be used as a final output (e.g., for display to a user). However, in some cases, this improved reconstruction is used as a synthetic prior.

Blocks 622, 624, 626, 628 can be used to correct for attenuation corresponding to materials outside of the field of view.

At block 622, a synthetic prior (e.g., the synthetic prior from block 620) is forward projected to obtain a new projection dataset. Any suitable forward imaging operator can be used, such as those described herein.

At block 624, an attenuation representation is determined using the received imaging data from block 602 and the new projection dataset from block 622. Determining the attenuation representation can include subtracting the new projection dataset (e.g., a reconstruction thereof) from the received imaging data (e.g., a reconstruction). The residual from this subtraction includes data that is present in the new projection dataset but not in the received imaging data, which is a representation of the attenuation occurring due to materials outside the field of view. This representation can be referred to as the outside-field-of-view (OFOV) attenuation representation.

At block 626, this OFOV attenuation representation can be denoised to obtain a denoised OFOV attenuation representation, which more accurately represents the attenuation from outside the of field of view as it does not include other spurious signal from the received imaging data.

At block 628, synthetic imaging data based on the synthetic prior can be output. The denoised OFOV attenuation representation from block 626 can be added to the new projection dataset to achieve a new projection dataset with attenuation. Then, the new projection dataset with attenuation outside the FOV can be back-projected into the image domain. Then, the estimated noise field from block 604 can be added to this back-projection. The resultant imaging data can be used as the synthetic imaging data.

The synthetic imaging data from block 628 can then be used for further purposes, such as to train a second imaging restoration process using this synthetic imaging data as input and the synthetic prior as the desired output. The trained second imaging restoration process can then be applied to the received imaging data to generate restored imaging data.

While described with certain blocks in a certain order, the process 600, the sequence may be altered without departing from the scope of the present disclosure. For example, some blocks may be performed in parallel or in a different sequence that does not materially affect the function of the process 600. In other examples, different components of an example device or system that implements the process 600 may perform functions at substantially the same time or in a specific sequence. In some cases, process 600 may include fewer blocks or additional blocks. For example, in some cases, especially when the imaging is a full field of view imaging, blocks 622, 624, 626, and 628 may be removed and the reconstruction resulting from block 620 can be used as a final output (e.g., for display to a user or for further analysis) or as a synthetic prior to which a different second imaging restoration process is applied (e.g., as described with reference to process 300 of FIG. 3).

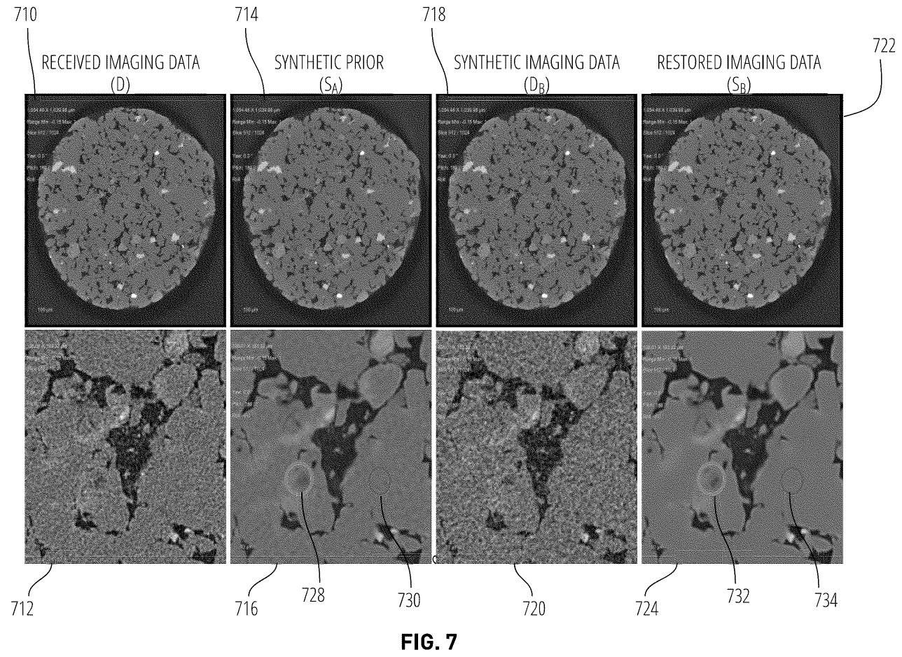

FIG. 7 is a set of images depicting various sets of imaging data of sandstone rock, according to certain aspects of the present disclosure. The imaging data depicted in FIG. 7 was acquired from X-ray microscopy of sandstone rock. Each column depicts a full field of view image at the top and a zoomed-in portion below. The imaging data depicted in FIG. 7 represents different imaging data at different times in a synthetic prior image restoration method, such as process 300 of FIG. 3. Specifically, the imaging data depicted in FIG. 7 was generated through processes the same as or similar to processes 400, 500 of FIG. 4 and FIG. 5, respectively.

The full field received imaging data 710 depicts an image of the sandstone rock as acquired from the imager, prior to any restoration processes. As seen in the zoomed received imaging data 712, the image quality is somewhat low, with much noise and various artifacts.

Applying a first imaging restoration process to the received imaging data results in the full field synthetic prior 714. As seen in the full field synthetic prior 714 and the zoomed synthetic prior 716, the image quality is much improved from the full field received imaging data 710 and zoomed received imaging data 712. The noise is greatly reduced and some artifacts are reduced or eliminated. However, certain artifacts remain (e.g., residual artifacts 730) and certain structures are not clearly depicted (e.g., faint structure 728).

As described herein, a forward imaging operator is applied to the synthetic prior to generate the full field synthetic imaging data 718. The full field synthetic imaging data 718 and zoomed synthetic imaging data 720 appears somewhat similar to the full field received imaging data 710 and zoomed received imaging data 712, respectively, which is to be expected, as the synthetic imaging data is intended to be used as an input to train a second imaging restoration process.

A second imaging restoration process can be trained using the full field synthetic prior 714 and full field synthetic imaging data 718 (or at least using portions thereof, such as the zoomed synthetic prior 716 and the zoomed synthetic imaging data 720). Once trained, the second imaging restoration process can be applied to the full field received imaging data 710 to generate the full field restored imaging data 722.

As seen in FIG. 7, the full field restored imaging data 722 and the zoomed restored imaging data 724 is not only much improved over the full field received imaging data 710 and zoomed received imaging data 712, but also substantially improved over the full field synthetic prior 714 and zoomed synthetic prior 716. The full field restored imaging data 722 has further reduced noise and artifacts, while retaining better clarity of small structures. For example, improved structure 732 is much more visible in the zoomed restored imaging data 724 than the faint structure 728 in the zoomed synthetic prior 716. Likewise, the residual artifacts 730 from the full field synthetic prior 714 have been much improved in the zoomed restored imaging data 724, resulting in improved residual artifacts 734 (e.g., no artifacts or reduced artifacts).

Overall, the amount of recovery achieved by the full field restored imaging data 722 is substantially improved over that of the full field synthetic prior 714.

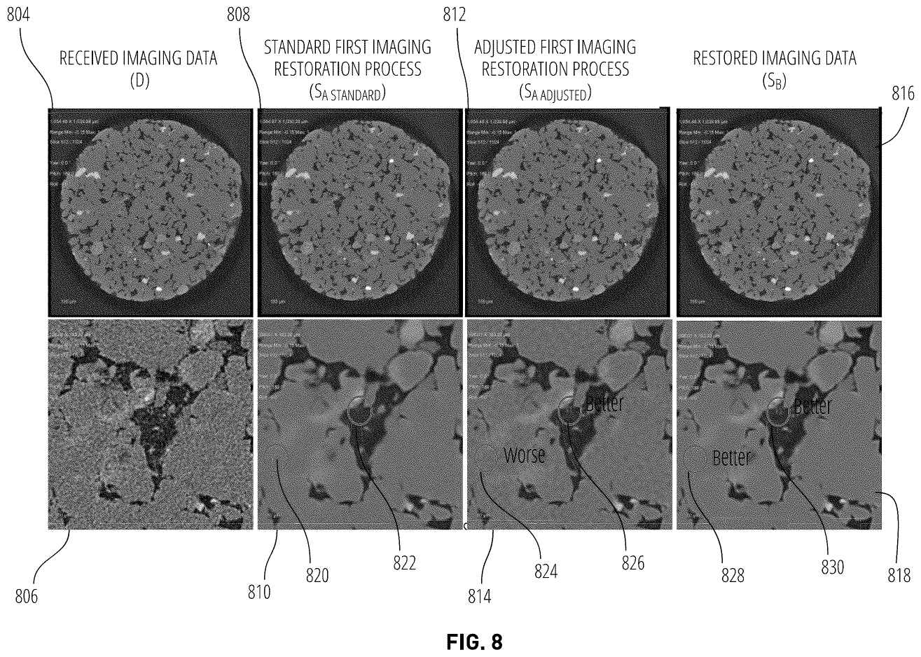

FIG. 8 is a set of images depicting various sets of imaging data of sandstone rock, according to certain aspects of the present disclosure. The imaging data depicted in FIG. 8 was acquired from X-ray microscopy of sandstone rock. Each column depicts a full field of view image at the top and a zoomed-in portion below.

The full field received imaging data 804 depicts an image of the sandstone rock as acquired from the imager, prior to any restoration processes. As seen in the zoomed received imaging data 806, the image quality is somewhat low, with much noise and various artifacts.

The full field synthetic prior using standard first imaging restoration process 808 is a synthetic prior obtained using a standard first imaging restoration process, such as that used to generate the full field synthetic prior 714 of FIG. 7. The standard first imaging restoration process is one that is intended to output an image suitable for final output, such as to be displayed to a user or used for substantive analysis. As seen in the zoomed synthetic prior using standard first imaging restoration process 810, the first imaging restoration process has been trained to achieve good looking results with reduced noise and certain artifacts (e.g., in the center of grains), such as at region 820, however is subject to over-smoothing of small features (e.g., small feature 822).

If the full field synthetic prior using standard first imaging restoration process 808 were used as the synthetic prior for generating synthetic imaging data and training a second imaging restoration process, the final restored imaging data that is output can be the same as or similar to zoomed restored imaging data 724 of FIG. 7.

However, since the synthetic prior is only used as intermediate imaging data according to certain aspects and features of the present disclosure, it is not otherwise be displayed to user or used for substantive analysis. Thus, in some cases, the first imaging restoration process used to generate the synthetic prior can be adjusted to produce intermediate imaging data that is more useful for the generation of synthetic imaging data and training of a second imaging restoration process, at the cost of visual or other degradations that may make the synthetic prior otherwise unsuitable for a final output.

The full field synthetic prior using adjusted first imaging restoration process 812 is a synthetic prior that has been obtained using an adjusted first imaging restoration process. In the example used with FIG. 8, the adjusted first imaging restoration process has an adjusted network structure and uses an asymmetric loss function rather than a symmetric loss function. As seen in the zoomed synthetic prior using adjusted first imaging restoration process 814, this synthetic prior gives results with worse residual artifacts (e.g., residual artifact 824) in the center of the grains (e.g., due to an increase in correlation between spurious signals between the subsets), but with the benefit of reducing the over-smoothing of small features (e.g., small feature 826).

Since the residual artifacts (e.g., residual artifact 824) of the synthetic prior are correlated with the synthetic imaging data, the second imaging restoration process does not learn to recover these residual artifacts. Thus, the residual artifacts are not present in the full field restored imaging data 816 and zoomed restored imaging data 818, as seen from the artifact-free region 828.

Further, the presence of not-over-smoothed, distinct small features (e.g., small feature 830) in the zoomed restored imaging data 818 means that the feature distribution present in the full field synthetic prior using adjusted first imaging restoration process 812 more closely correlates to the true structure feature distribution, resulting in better feature recover than if the standard first imaging restoration process were used in the creation of the synthetic prior.

Many other such adjustments can be made to the workflow (e.g., process 300 of FIG. 3) to achieve a synthetic prior that closely correlates to the structure function, such as loss weighting in the final recovery network, weighting the loss computed in regions of high gradient, or corresponding to small features, higher than the loss in the center of large uniform objects, potentially giving better recovery of small features.

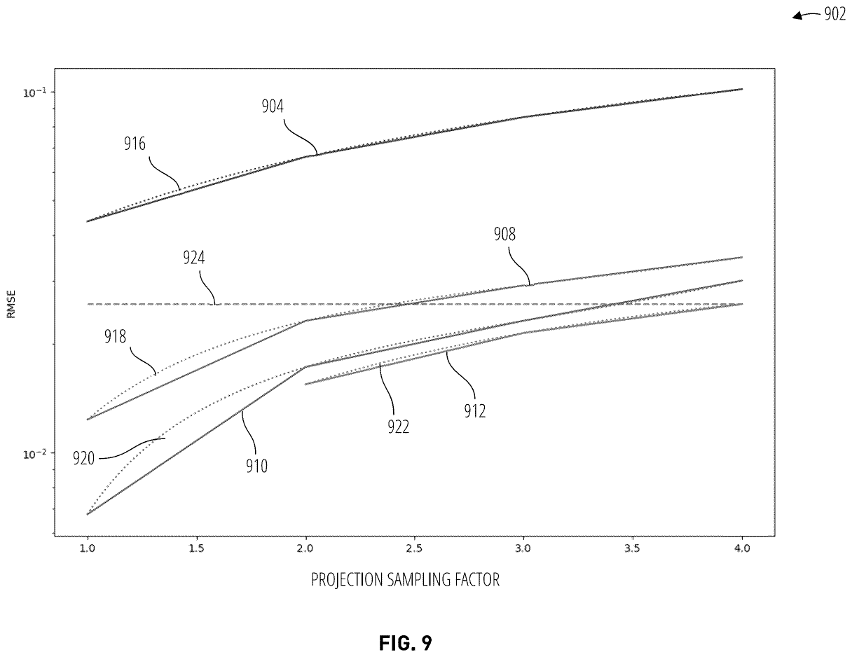

FIG. 9 is a chart 902 depicting the performance of various imaging restoration processes, according to certain aspects of the present disclosure. Chart 902 depicts the performance of various imaging restoration processes at various projection sampling factors. The projection sampling factors are based on a full 1601 projection dataset, such that a projection sampling factor of 1 corresponds to a reconstruction that is provided all 1601 projections to use, whereas a projection sampling factor of 2 corresponds to a reconstruction that is provided 800 projections to use. The performance of each imaging restoration process is depicted as the root mean square error (RMSE) of the reconstructed dataset (e.g., restored imaging data), computed relative to a best structure function (S) estimate (e.g., best restored imaging data) found using synthetic prior reconstruction using the full projection set of 1601 projections (e.g., a projection sampling of 1).

Line 904 represents the Feldkamp, Davis and. Kress (FDK) reconstruction algorithm, which always shows a high RMSE due to its extensive noise and artifacts. Dotted line 916 is a fitted polynomial regression curve for line 904.

Line 908 is an existing imaging restoration process that relies on deep learning based X-ray reconstruction. Line 908 shows significantly better fidelity of reconstruction than line 904. Dotted line 918 is a fitted polynomial regression curve for line 908.

Line 910 is a synthetic prior reconstruction (e.g., as described with reference to process 300 of FIG. 3) that uses the imaging restoration process from line 908 as its first imaging restoration process, to generate its synthetic prior, which is then used to generate synthetic imaging data and train a second imaging restoration process, which is then used to output the restored imaging data from the received imaging data. Dotted line 920 is a fitted polynomial regression curve for line 910. It can be seen that line 910 provides significantly better fidelity of reconstruction than line 904 and line 908.

Line 912 is a synthetic prior reconstruction that uses an adjusted imaging restoration process, such as that described with reference to FIG. 8, to generate its synthetic prior. Specifically, the imaging restoration process used for line 912 is the imaging restoration process from line 908 with a modified loss and network structure. Dotted line 922 is a fitted polynomial regression curve for line 912. It can be seen that line 912 provides significantly better fidelity of reconstruction than line 904 and line 908, and slightly better fidelity of reconstruction than line 910.

The dashed line 924 is an indication of the improvement achieved. Dashed line 924 is set at the RMSE for line 912 at a projection sampling factor of 4 (e.g., 400 projections). The position on the X axis of the intersection of this dashed line 924 with the other lines corresponds to the number of projections that these other techniques require to achieve the same RMSE as synthetic prior with an adjusted network and loss achieves at 400 projections. For example, line 908 shows that the existing imaging restoration process would require use of approximately 670 projections to achieve the same results as the synthetic prior (with adjusted network and loss) can achieve with 400 projections, an effective speed increase of 67%.

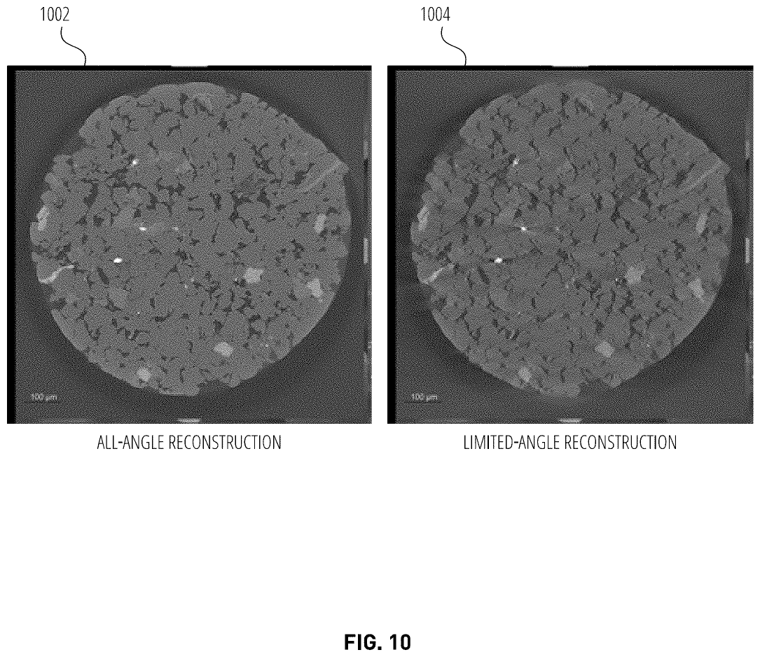

FIG. 10 is a set of image reconstructions for a sample, according to certain aspects of the present disclosure. The all-angle reconstruction 1002 is a reconstruction of a sample generated by using all projection angles (e.g., 0-180 degrees). The limited-angle reconstruction 1004 is a reconstruction of the same sample generated by using a limited set of projection angles (e.g., 30-150 degrees). As seen in limited-angle reconstruction 1004, streaks and anomalous reconstructed attenuations are present where none appear in the all-angle reconstruction 1002.

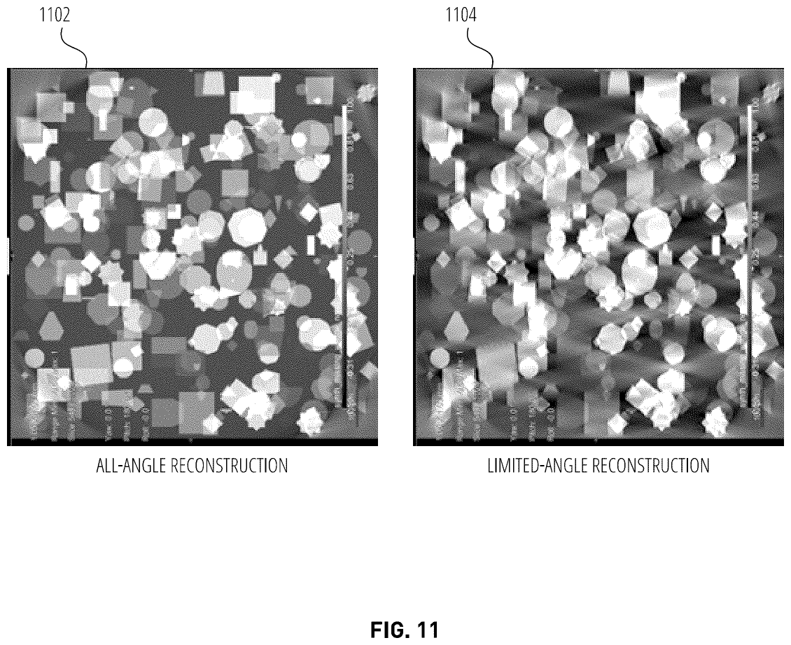

FIG. 11 is a set of image reconstructions for a training volume, according to certain aspects of the present disclosure. The training volume can be any suitable volume containing various structures that can somewhat approximate the intended sample for which imaging restoration is intended. For example, for an imaging of sandstone, a volume of arbitrary objects (e.g., spheres, cubes, other shapes) can be generated. This training volume can then be forward projected into both an all-angle dataset and a limited-angle dataset. Specifically, the limited set of projection angles used for the limited-angle dataset the same limited set of projection angles available in the received imaging data (e.g., the projection angles associated with limited-angle reconstruction 1004 of FIG. 10).

The all-angle reconstruction 1102 is a reconstruction of the training volume generated by using the all-angle dataset. The limited-angle reconstruction 1104 is a reconstruction of the same training volume generated by using the limited-angle dataset.

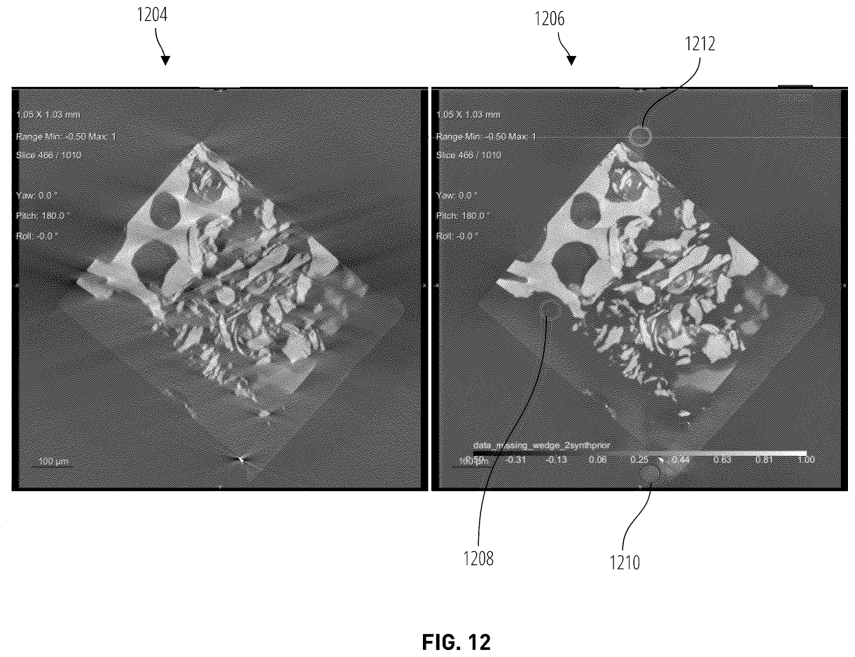

FIG. 12 is a set of image reconstructions for a sample, according to certain aspects of the present disclosure. The initial reconstruction 1204 is a reconstruction of received imaging data corresponding to imaging of a sample. Here, the received imaging data contains projections from only a limited set of angles (e.g., 30-150 degrees). As such, the initial reconstruction 1204 contains various artifacts seen in other limited-angle reconstructions (e.g., limited-angle reconstruction 1004 of FIG. 10).

The synthetic prior 1206 is a corrected version of the initial reconstruction 1204 after application of an imaging restoration process that has been trained using a synthetic training volume (e.g., the training volume described with reference to FIG. 11). The synthetic prior 1206 exhibits a significant reduction in the missing wedge artifact.

However, some further artifacts remain in the synthetic prior 1206, such as anomalously non-uniform reconstructed absorption values (e.g., region 1210) and anomalously high absorption values (ghosting) around the edge of features (e.g., region 1212) and around high-intensity grains (e.g., region 1210). This synthetic prior 1206 can be further used as described with reference to process 600 of FIG. 6.

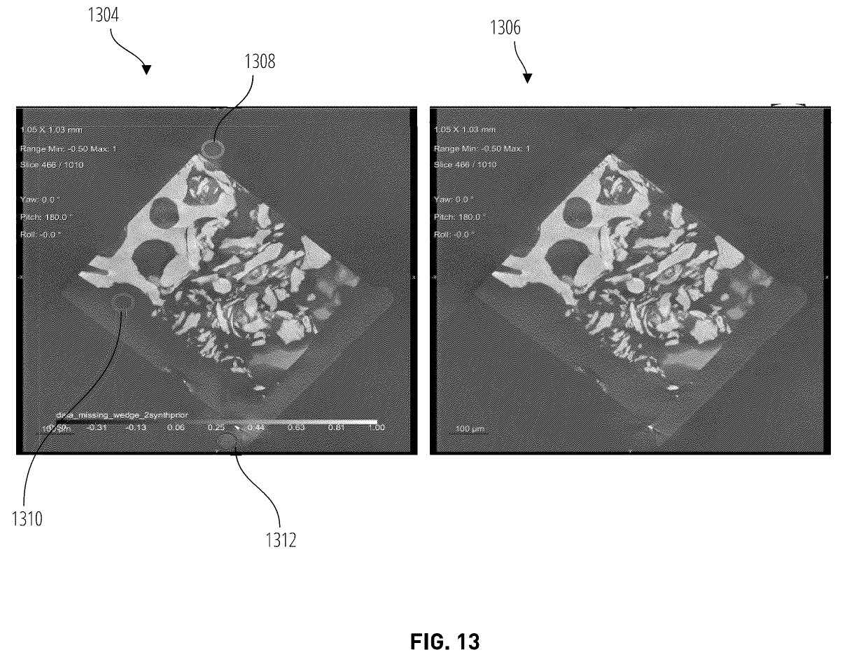

FIG. 13 is a set of image reconstructions for a sample (e.g., the sample of FIG. 12), according to certain aspects of the present disclosure. The synthetic prior 1304 can be the same as or similar to the synthetic prior 1206 of FIG. 12. As seen in the synthetic prior 1304, various artifacts remain, such as anomalously non-uniform reconstructed absorption values (e.g., region 1310) and anomalously high absorption values (ghosting) around the edge of features (e.g., region 1308) and around high-intensity grains (e.g., region 1312).

The restored imaging data 1306 is imaging data that has been processed by a second imaging restoration process that was trained based on the synthetic prior 1206 and synthetic imaging data generated from the synthetic prior (e.g., generated according to process 600 of FIG. 6).

As seen in the restored imaging data 1306, the anomalously low reconstructed values are reduced, and the ghosting near the edge of features and around high-intensity grains is removed.

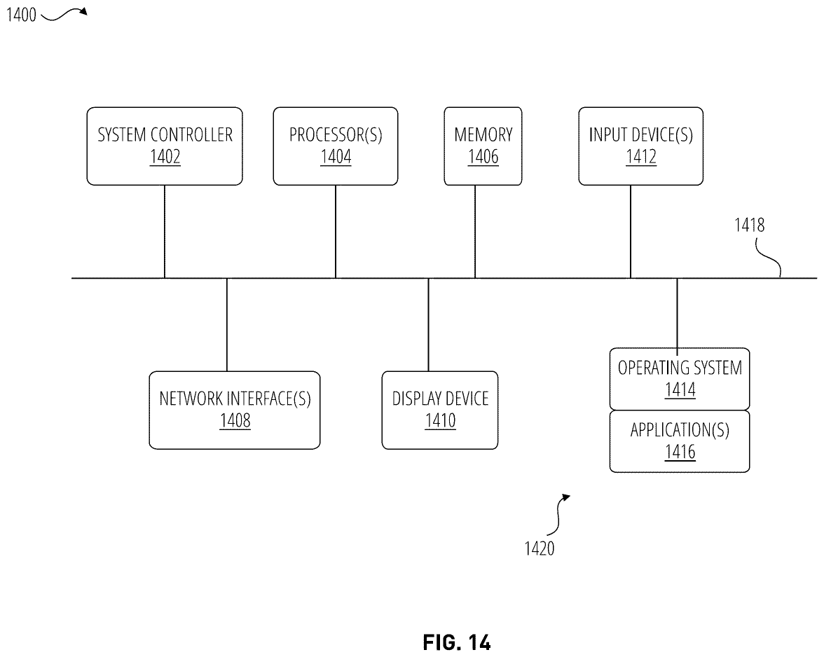

FIG. 14 is a block diagram of an example system architecture 1400 for implementing features and processes of the present disclosure, such as those presented with reference to processes 300, 400, 500, 600, of FIGS. 3, 4, 5, 6, respectively. The features and processes disclosed herein can be implemented using one or multiple instances of 1400. The system architecture 1400 can be used to implement a server (e.g., a cloud-accessible server), a user device (e.g., a smartphone or personal computer), or any other suitable device for performing some or all of the aspects of the present disclosure. The system architecture 1400 can be implemented on any electronic device that runs software applications derived from compiled instructions, including without limitation personal computers, servers, imagers (e.g., X-ray microscopes), smart phones, electronic tablets, game consoles, email devices, and the like. In some implementations, the system architecture 1400 can include one or more processors 1404, one or more input devices 1412, one or more display devices 1410, one or more network interfaces 1408, and one or more computer-readable media 1420. Each of these components can be coupled by bus 1418.

Display device 1410 can be any known display technology, including but not limited to display devices using Liquid Crystal Display (LCD) or Light Emitting Diode (LED) technology. Processor(s) 802 can use any known processor technology, including but not limited to graphics processors and multi-core processors. Input device 1412 can be any known input device technology, including but not limited to a keyboard (including a virtual keyboard), mouse, track ball, and touch-sensitive pad or display. In some cases, audio inputs can be used to provide audio signals, such as audio signals of an individual speaking. Bus 1418 can be any known internal or external bus technology, including but not limited to ISA, EISA, PCI, PCI Express, NuBus, USB, Serial ATA or FireWire.

Computer-readable medium 1420 can be any medium that participates in providing instructions to processor 1404 for execution, including without limitation, non-volatile storage media (e.g., optical disks, magnetic disks, flash drives, etc.) or volatile media (e.g., SDRAM, ROM, etc.). The computer-readable medium (e.g., storage devices, mediums, and memories) can include, for example, a cable or wireless signal containing a bit stream and the like. However, when mentioned, non-transitory computer-readable storage media expressly exclude media such as energy, carrier signals, electromagnetic waves, and signals per se.

Computer-readable medium 1420 can include various instructions for implementing operating system 1414 and applications 1416 such as computer programs. The operating system 1414 can be multi-user, multiprocessing, multitasking, multithreading, real-time and the like. The operating system 1414 performs basic tasks, including but not limited to: recognizing input from input device 1412; sending output to display device 1410; keeping track of files and directories on computer-readable medium 1420; controlling peripheral devices (e.g., storage drives, interface devices, etc.) which can be controlled directly or through an I/O controller; and managing traffic on bus 1418. Computer-readable medium 1420 can include various instructions for implementing firmware processes, such as a BIOS. Computer-readable medium 1420 can include various instructions for implementing any of the processes described herein, including at least processes 300, 400, 500, 600 of FIGS. 3, 4, 5, 6, respectively.

Memory 1406 can include high-speed random access memory and/or non-volatile memory, such as one or more magnetic disk storage devices, one or more optical storage devices, and/or flash memory (e.g., NAND, NOR). The memory 1406 (e.g., computer-readable storage devices, mediums, and memories) can include a cable or wireless signal containing a bit stream and the like. However, when mentioned, non-transitory computer-readable storage media expressly exclude media such as energy, carrier signals, electromagnetic waves, and signals per se. The memory 1406 can store an operating system, such as Darwin, RTXC, LINUX, UNIX, OS X, WINDOWS, or an embedded operating system such as VxWorks.

System controller 1402 can be a service processor that operates independently of processor 1404. In some implementations, system controller 1402 can be a baseboard management controller (BMC). For example, a BMC is a specialized service processor that monitors the physical state of a computer, network server, or other hardware device using sensors and communicating with the system administrator through an independent connection. The BMC is configured on the motherboard or main circuit board of the device to be monitored. The sensors of a BMC can measure internal physical variables such as temperature, humidity, power-supply voltage, fan speeds, communications parameters and operating system (OS) functions.

The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language (e.g., Objective-C, Java), including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors or cores, of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

To provide for interaction with a user, the features can be implemented on a computer having a display device such as a CRT (cathode ray tube) or LCD (liquid crystal display) monitor for displaying information to the user and a keyboard and a pointing device such as a mouse or a trackball by which the user can provide input to the computer.

The features can be implemented in a computing system that includes a back-end component, such as a data server, or that includes a middleware component, such as an application server or an Internet server, or that includes a front-end component, such as a client computer having a graphical user interface or an Internet browser, or any combination thereof. The components of the system can be connected by any form or medium of digital data communication such as a communication network. Examples of communication networks include, e.g., a LAN, a WAN, and the computers and networks forming the Internet.

The computing system can include clients and servers. A client and server are generally remote from each other and typically interact through a network. The relationship of client and server arises by virtue of computer programs running on the respective computers and having a client-server relationship to each other.

One or more features or steps of the disclosed embodiments can be implemented using an application programming interface (API). An API can define one or more parameters that are passed between a calling application and other software code (e.g., an operating system, library routine, function) that provides a service, that provides data, or that performs an operation or a computation.

The API can be implemented as one or more calls in program code that send or receive one or more parameters through a parameter list or other structure based on a call convention defined in an API specification document. A parameter can be a constant, a key, a data structure, an object, an object class, a variable, a data type, a pointer, an array, a list, or another call. API calls and parameters can be implemented in any programming language. The programming language can define the vocabulary and calling convention that a programmer will employ to access functions supporting the API.

In some implementations, an API call can report to an application the capabilities of a device running the application, such as input capability, output capability, processing capability, power capability, communications capability, and the like.

Certain aspects and features of the present disclosure relate to methods, systems, and non-transitory computer-readable media for reconstructing computed tomography (CT) images from sparse-view imaging data using synthetic prior generation, spectral noise field synthesis, and throughput-conditioned machine learning models. In some instances, well-known structures and functions have not been shown or described in detail to avoid unnecessarily obscuring the description of the present disclosure.

In some implementations, the present disclosure addresses limitations of prior approaches to CT image reconstruction from sparse-view data. Conventional analytical reconstruction algorithms, such as filtered back-projection (FBP), are effective for full-view CT data but not effective for sparse-view CT data. They produce reconstructions with severe noise and artifacts when applied to sparse-view data having a reduced number of projection views. Deep learning-based methods have been developed for CT image reconstruction. But these methods generally focus on two-dimensional fan-beam CT or medical imaging applications. They do not robustly address noise reduction and sparse sampling artifact removal in three-dimensional cone-beam CT for industrial or high-throughput settings. Additionally, methods effective for full-view CT data are not optimized for the unique noise characteristics and artifact patterns introduced by sparse-view acquisition. The present disclosure provides a staged reconstruction framework. This framework combines analytical CT reconstruction, synthetic prior generation via grouped reconstructions, spectral noise field synthesis, and throughput-conditioned machine learning to enable high-quality image reconstruction from sparse-view CT data.

In some implementations, the present disclosure provides several contributions. First, the disclosure provides a method to generate a synthetic prior from statistically related reconstructions generated from grouped raw data. Second, the disclosure provides a spectral noise field synthesis technique that preserves local spectral characteristics of a difference field while regenerating phase structure to produce realistic training data. Third, the disclosure extends the reconstruction pipeline into a throughput-conditioned sparse-view setting, thus enabling robust noise and sparse sampling artifact reduction even when only a limited number of projection views are available.

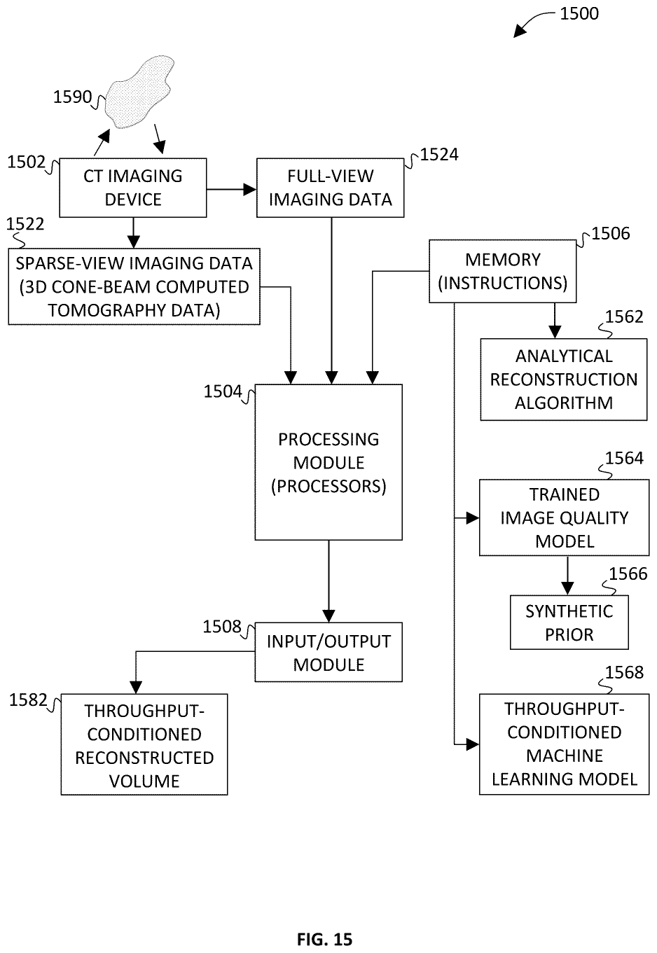

FIG. 15 illustrates a system 1500 for reconstructing computed tomography (CT) images from sparse-view imaging data 1522. The system 1500 includes a CT imaging device 1502. The CT imaging device 1502 may be a cone-beam CT system that is designed to acquire three-dimensional cone-beam computed tomography data. The CT imaging device 1502 is designed to acquire full-view imaging data 1524 and sparse-view imaging data 1522 of a subject 1590. The sparse-view imaging data 1522 is acquired at a throughput-conditioned acquisition setting. Additionally, the sparse-view imaging data 1522 has fewer projection views than the full-view imaging data 1524.

The system 1500 further includes a processing module 1504. The processing module 1504 includes one or more processors and a memory 1506. The memory 1506 is coupled to the processing module 1504. Additionally, the memory 1506 stores instructions. When executed by the processing module 1504, the instructions cause the system 1500 to perform the reconstruction methods described herein. The memory 1506 further stores an analytical reconstruction algorithm 1562, a trained image quality model 1564, a synthetic prior 1566, and a throughput-conditioned machine learning model 1568. The synthetic prior 1566 is generated based on full-view imaging data 1524. Moreover, the synthetic prior 1566 is used to generate the trained image quality model 1564. The system 1500 may further include an input/output module 1508. For example, the system 1500 outputs a throughput-conditioned reconstructed volume 1582 with reduced noise and sparse sampling artifacts via the input/output module 1508.

In some implementations, the CT imaging device 1502 comprises a three-dimensional cone-beam CT system. The CT imaging device 1502 may acquire the sparse-view imaging data 1522 in a high-throughput industrial inspection setting, wherein the subject 1590 comprises an industrial sample. Sparse-view acquisition is common in industrial and scientific CT applications where increased scan throughput or reduced scan time is desirable.

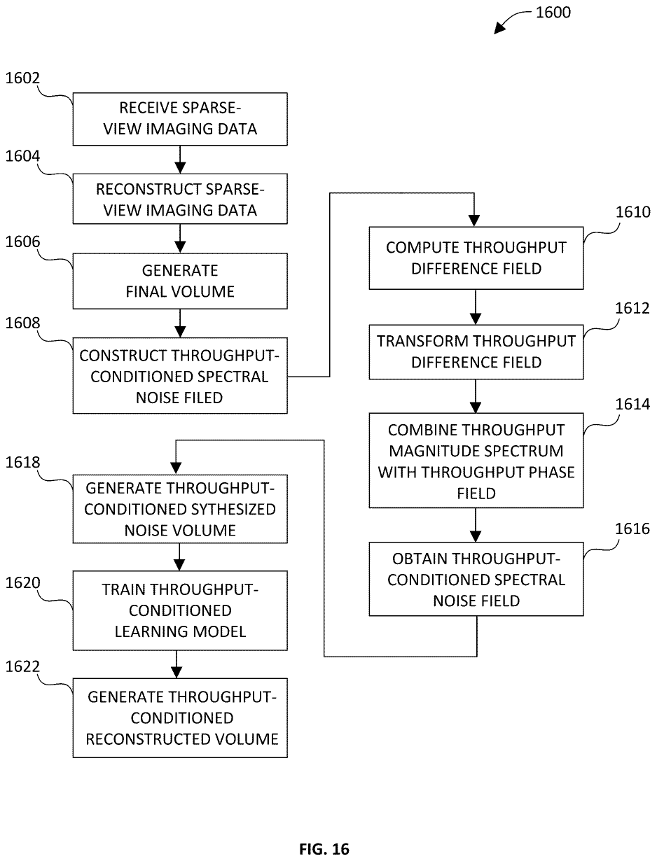

FIG. 16 illustrates a process 1600 for reconstructing an image from sparse-view computed tomography (CT) imaging data. The process 1600 may be performed using any suitable hardware, such as the system 1500 of FIG. 15.

At block 1602, sparse-view imaging data of a subject is received. The sparse-view imaging data is acquired by a CT imaging device at a throughput-conditioned acquisition setting. The sparse-view imaging data has fewer projection views than full-view imaging data of the subject. The throughput-conditioned acquisition setting defines a reduction factor in a number of projection views relative to the full-view imaging data.

At block 1604, the sparse-view imaging data is reconstructed using an analytical reconstruction algorithm to produce a sparse-view reconstruction. In some implementations, the analytical reconstruction algorithm comprises filtered back-projection (FBP). The FBP generates a reconstruction of the sparse-view data that achieves significant reduction of the noise and sparse sampling artifacts inherent with the reduced number of projection views.

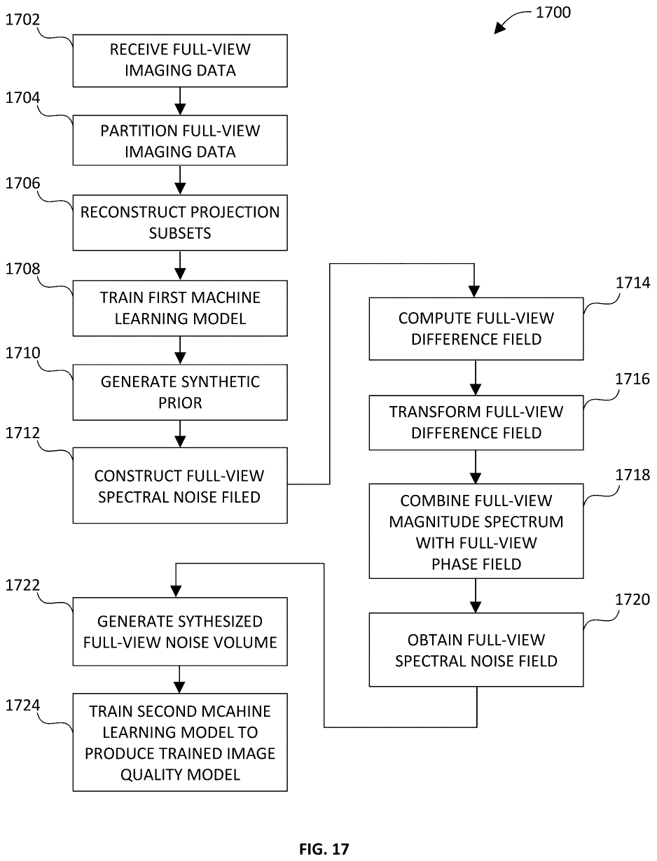

At block 1606, a final reconstructed volume of the subject is generated. The final reconstructed volume is generated from a trained image quality model that is created using a synthetic prior. The creation of the trained image quality model is described in further detail with reference to FIG. 17.

At block 1608, a throughput-conditioned spectral noise field is constructed. Constructing the throughput-conditioned spectral noise field includes the following sub-steps.

At block 1610, a throughput difference field is computed between the sparse-view reconstruction and the final reconstructed volume.

At block 1612, the throughput difference field is transformed into a throughput frequency domain to obtain a throughput magnitude spectrum.

At block 1614, the throughput magnitude spectrum is combined with a throughput phase field to produce a synthesized throughput frequency-domain representation.

At block 1616, an inverse frequency-domain transform is applied to the synthesized throughput frequency-domain representation to obtain the throughput-conditioned spectral noise field. The purpose of this spectral noise field synthesis is to preserve the local spectral envelope of the difference field while regenerating its phase structure in a controlled way, thereby producing a realistic stochastic training component whose frequency statistics remain tied to the measured difference field.

At block 1618, a throughput-conditioned synthesized noise volume is generated by adding the throughput-conditioned spectral noise field to the final reconstructed volume.

At block 1620, a throughput-conditioned machine learning model is trained using the throughput-conditioned synthesized noise volume as input and the final reconstructed volume as a target.

At block 1622, the throughput-conditioned machine learning model is applied to the sparse-view reconstruction to generate a throughput-conditioned reconstructed volume. The throughput-conditioned reconstructed volume achieves reduced noise and sparse sampling artifacts relative to the sparse-view reconstruction.

FIG. 17 illustrates a process 1700 for creating a trained image quality model using full-view imaging data. The process 1700 provides the creation of the trained image quality model referenced in block 1606 of FIG. 16.

At block 1702, full-view imaging data of the subject is received. The full-view imaging data is acquired by the CT imaging device. Moreover, the full-view imaging data has a full complement of projection views.

At block 1704, the full-view imaging data is partitioned into a first projection subset and a second projection subset. In some implementations, the projection data are divided into two groups of approximately equal size.

At block 1706, the first projection subset and the second projection subset are independently reconstructed using the analytical reconstruction algorithm to produce a first grouped reconstruction and a second grouped reconstruction, respectively. Each grouped reconstruction is produced using filtered back-projection (FBP) on its respective subset of the projection data. The two grouped reconstructions serve as the training pair for the first learning phase.

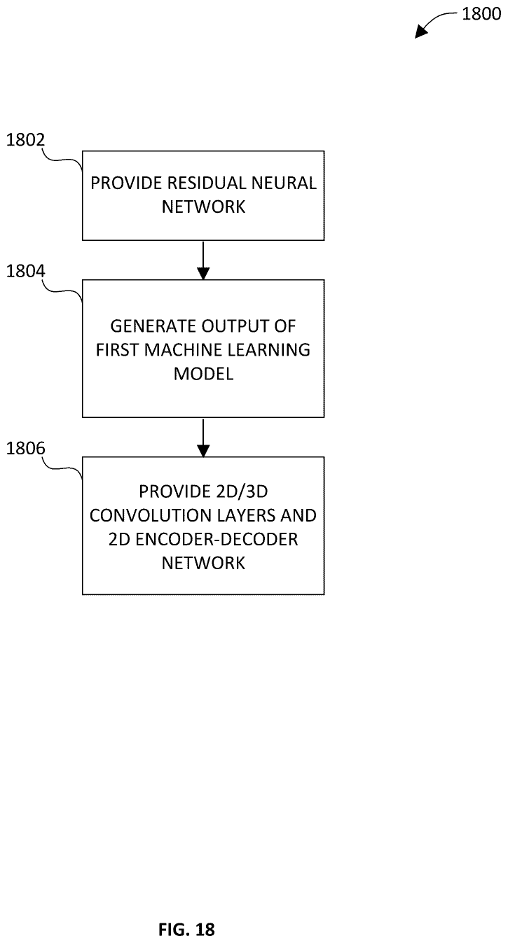

At block 1708, a first machine learning model is trained on the first grouped reconstruction and the second grouped reconstruction to learn a synthetic prior model. The role of the first machine learning model is to learn a synthetic prior model that captures the stable structural content shared across independently reconstructed subsets of the same scan. The architecture and training of the first machine learning model are described in further detail with reference to FIG. 18.

At block 1710, the trained first machine learning model is applied to a baseline reconstruction. The baseline reconstruction is derived from the full-view imaging data to generate a synthetic prior. The baseline reconstruction is produced by applying the analytical reconstruction algorithm to the complete full-view imaging data. The synthetic prior represents a denoised structural estimate of the subject.

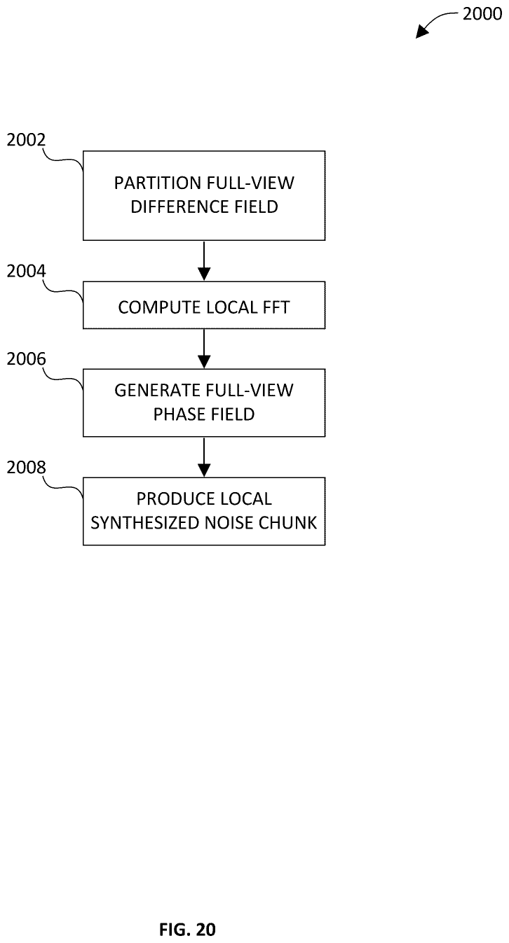

At block 1712, a full-view spectral noise field is constructed. The construction of the full-view spectral noise field is described in further detail with reference to FIG. 20. Specifically, the construction of the full-view spectral noise field synthesis includes: (i) computing a full-view difference field between the baseline reconstruction and the synthetic prior at block 1714, (ii) transforming the full-view difference field into a full-view frequency domain at block 1716, (iii) combining the full-view magnitude spectrum with a full-view phase field at block 1718, and (iv) obtaining the full-view spectral noise field at block 1720.

At block 1722, a synthesized full-view noise volume is generated by adding the full-view spectral noise field to the synthetic prior. This synthesized noise volume bridges the first and second learning phases. The first phase provides a denoised structural prior. Additionally, the spectral remapping stage supplies a realistic stochastic component. And the second phase then learns to reconstruct from this noise-conditioned pairing.

At block 1724, a second machine learning model is trained using the synthesized full-view noise volume as input and the synthetic prior as a target to produce the trained image quality model.

FIG. 18 illustrates a process 1800 for providing a residual neural network architecture to a machine learning model. At block 1802, a residual neural network is provided to the first machine learning model. The residual neural network is configured such that its output can be expressed as

$$\hat{y} = x + {f}_{θ} ( x ) ,$$

where x is an input reconstruction and f_{θ}(x) is the learned correction. In other words, the network does not synthesize the reconstruction from scratch. It learns how to improve an existing FBP reconstruction input.

At block 1804, the output of the first machine learning model is generated based on the learned correction. The residual architecture ensures that the model learns only the difference between the input and the desired output, which stabilizes training and improves convergence.

At block 1806, the residual neural network is provided with two-dimensional, three-dimensional convolution layers, and a two-dimensional encoder-decoder network. Specifically, the residual neural network comprises: (i) an initial three-dimensional convolution layer that extracts inter-slice features from a stack of adjacent input slices, (ii) a two-dimensional encoder-decoder network that processes the extracted features at multiple resolution levels, and (iii) a final three-dimensional output convolution layer that produces a volumetric output. The initial three-dimensional convolution extracts joint inter-slice features. Those features are reorganized into the two-dimensional encoder-decoder path with multiple resolution levels. The final three-dimensional output convolution brings the prediction back into volumetric form.

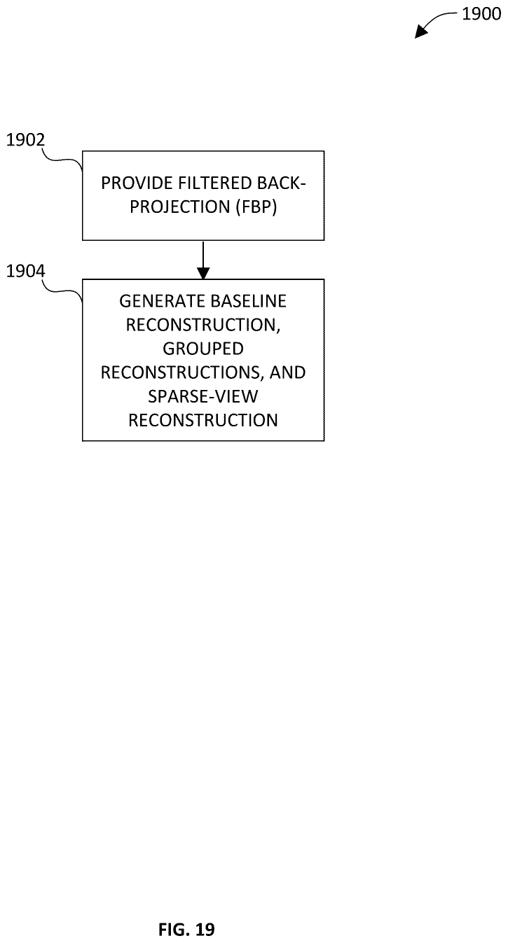

FIG. 19 illustrates a process 1900 for generating reconstructions using filtered back-projection (FBP).

At block 1902, the filtered back-projection (FBP) is provided as the analytical reconstruction algorithm. A standard analytical description of the configured FBP stage is

$${q}_{θ} ( s ) = ( {p}_{θ} \cdot h ) ( s ) ,$$

where p_{θ}(s) is the projection at view angle θ and h is the ramp filter. The reconstructed image can then be written in the usual FBP form.

At block 1904, the baseline reconstruction, the first grouped reconstruction, the second grouped reconstruction, and the sparse-view reconstruction are each generated using filtered back-projection. In some implementations, the FBP stage is used repeatedly: first to obtain two grouped reconstructions for training, then to obtain the full baseline reconstruction, and later again to obtain the throughput-conditioned sparse-view reconstruction.

FIG. 20 illustrates a process 2000 for constructing a full-view spectral noise field.

At block 2002, the full-view difference field is partitioned into local volumetric chunks. In some implementations, each local volumetric chunk has dimensions of 64×64×64 voxels. The difference field is padded along a slice direction to a multiple of 64 voxels prior to partitioning.

At block 2004, a local fast Fourier transform (FFT) is computed to generate a local magnitude spectrum for each chunk. The magnitude spectrum captures the local spectral envelope of the noise in the difference field.

At block 2006, the full-view phase field is generated for each chunk. The phase field may be generated by one of two methods: (i) generating randomized phases using a uniform random number generator, or (ii) computing an auto-phase field derived from a frequency-domain representation of a random noise field seeded by a local spatially variant standard deviation of the full-view difference field. The purpose of regenerating the phase structure is to produce decorrelated noise realizations while preserving the local frequency statistics of the original difference field.

At block 2008, the local magnitude spectrum is combined with the full-view phase field, and an inverse FFT is applied to produce a local synthesized noise chunk for each chunk. If chunk scaling is enabled, each synthesized chunk is further rescaled so that its standard deviation matches that of the corresponding chunk of the original difference field. The reconstructed spectral component for each chunk is

$$\tilde{V} ( k ) = A ( k ) \cdot {e}^{j ∅ ( k )} ,$$

where the magnitude spectrum

$$A ( k ) = \vert F \{ {υ}_{nf} ( k ) \} ( k ) \vert ,$$

and the synthesized spatial-domain noise field is

$$\tilde{υ} ( k ) = \text{?} \{ {F}^{- 1} ( \tilde{V} ) \} .$$

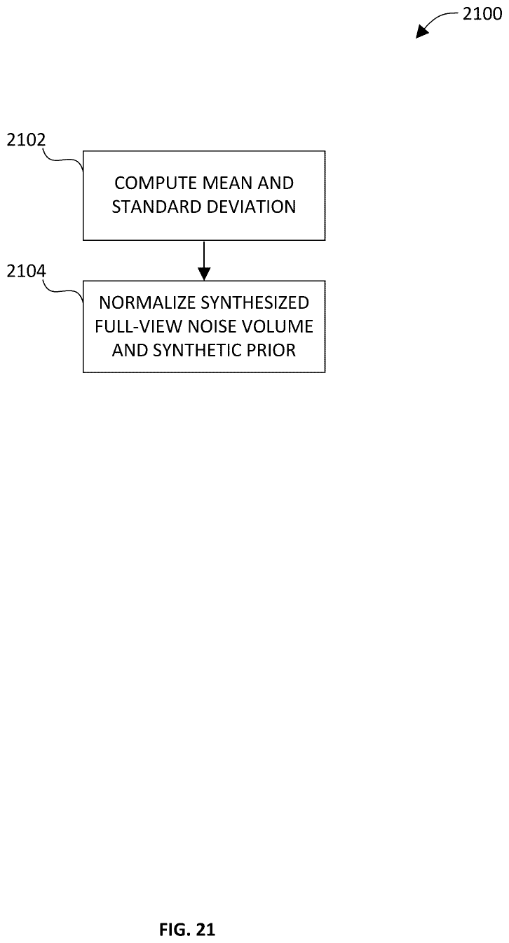

FIG. 21 illustrates a process 2100 for normalizing training inputs using computed mean and standard deviation.

At block 2102, a mean and a standard deviation of the synthesized full-view noise volume are computed.

At block 2104, both the synthesized full-view noise volume and the synthetic prior are normalized using the computed mean and standard deviation prior to training the second machine learning model. If the noise input has mean and standard deviation a, then both input and target are normalized as

$${x}^{′} = \frac{x - μ}{σ} ,$$

$${y}^{′} = \frac{y - μ}{σ} .$$

This normalization is a specific part of the second learning phase and ensures that the training operates on standardized data.

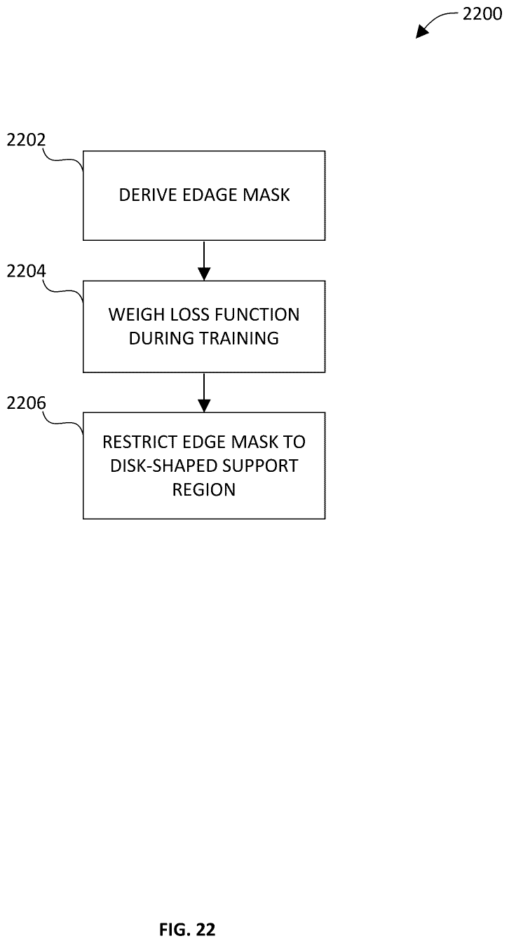

FIG. 22 illustrates a process 2200 for deriving an edge mask and applying edge-weighted loss during training.

At block 2202, an edge mask is derived from the synthetic prior by computing a magnitude of a Gaussian-smoothed residual of the synthetic prior. The edge mask identifies structurally informative edge regions within the volume.

At block 2204, a loss function is weighted during training such that regions identified by the edge mask receive higher loss weights than non-edge regions. This edge-weighted loss ensures that the model places stronger emphasis on preserving structural detail during training.

At block 2206, the edge mask is optionally restricted to a disk-shaped support region when a circular field-of-view constraint is active. In some implementations, the second machine learning model can be trained either over the default volume support or with stronger emphasis on structurally informative edge regions.

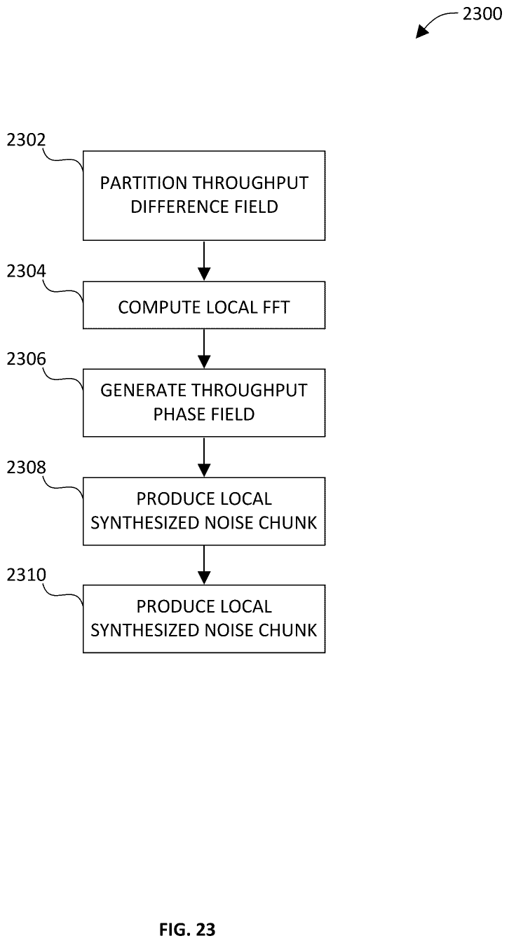

FIG. 23 illustrates a process 2300 for constructing a throughput-conditioned spectral noise field.

At block 2302, the throughput difference field is partitioned into local volumetric chunks. The throughput difference field is the difference between the sparse-view reconstruction and the final reconstructed volume.

At block 2304, for each chunk, a local fast Fourier transform (FFT) is computed to generate a local magnitude spectrum of the throughput difference field.

At block 2306, for each chunk, the throughput phase field is generated by one of: (i) generating randomized phases using a uniform random number generator, or (ii) computing an auto-phase field derived from a frequency-domain representation of a random noise field seeded by a local spatially variant standard deviation of the throughput difference field.

At block 2308, for each chunk, the local magnitude spectrum is combined with the throughput phase field, and an inverse FFT is applied to produce a local synthesized noise chunk.

At block 2310, the local synthesized noise chunks are assembled to produce the throughput-conditioned spectral noise field.

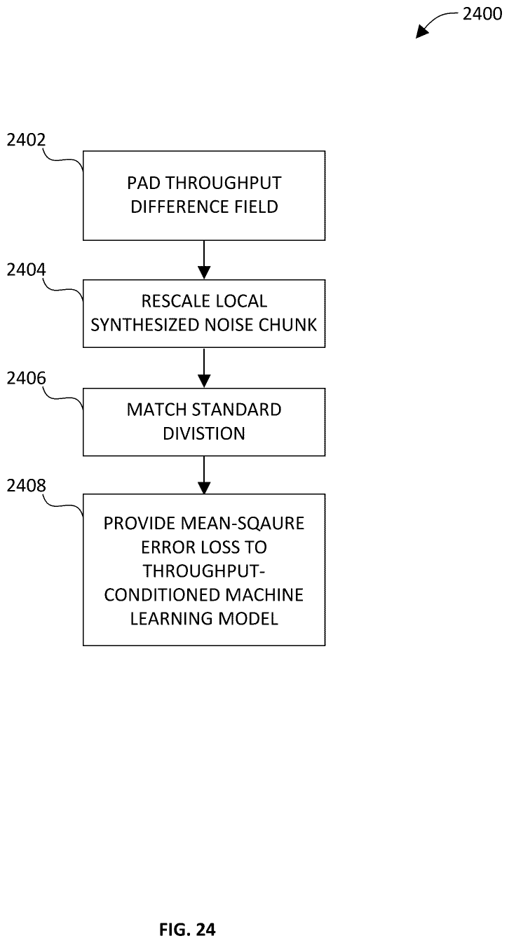

FIG. 24 illustrates a process 2400 for padding, rescaling, and applying a mean-squared error loss to a throughput-conditioned machine learning model.

At block 2402, the throughput difference field is padded along a slice direction to a multiple of 64 voxels prior to partitioning into local volumetric chunks. Each local volumetric chunk has dimensions of 64×64×64 voxels.

At block 2404, the local synthesized noise chunk is rescaled.

At block 2406, the standard deviation of each local synthesized noise chunk is matched to the standard deviation of a corresponding chunk of the throughput difference field. This rescaling ensures that the synthesized noise has locally consistent statistical properties with the measured difference.

At block 2408, a mean-squared error loss is provided to the throughput-conditioned machine learning model during training. The mean-squared error loss is defined as

$${L}_{MSE} = \frac{1}{N} {\sum }_{i = 1}^{N} {( {\hat{y}}_{i} - {y}_{i} )}^{2} ,$$

where ŷᵢ is a predicted output and yᵢ is a corresponding target. The training objective used across the learning phases is the mean-squared error, which measures the average squared difference between the predicted output and the corresponding target.

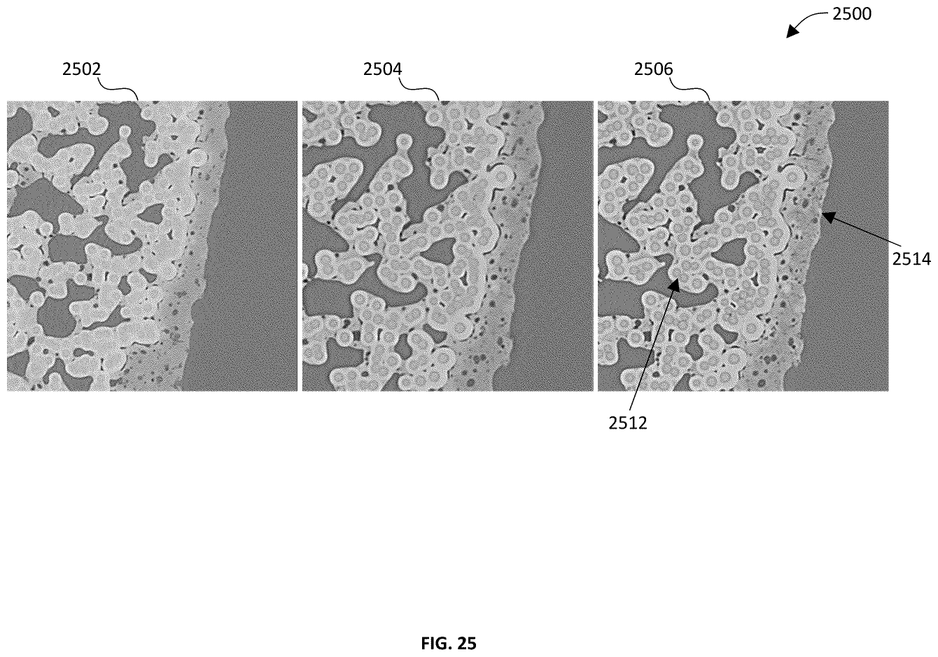

FIG. 25 depicts example reconstruction results comparing various methods for sparse-view CT imaging. The results compare outputs from different reconstruction approaches applied to sparse-view CT data. In some implementations, the comparison includes results from prior throughput-conditioned reconstruction methods, standard full-view image clarity reconstruction, and the throughput-conditioned image clarity reconstruction of the present disclosure. The throughput-conditioned reconstruction of the present disclosure demonstrates reduced noise and sparse sampling artifacts while maintaining structural fidelity, particularly in high-throughput industrial imaging scenarios.

Specifically, a set of images depicts various sparse-view imaging data 2500 of bin1 ceramic composite. The imaging data depicted in FIG. 25 was acquired from X-ray microscopy of the bin1 ceramic composite. Each image depicts a 4× sparse view at the top of the bin1 ceramic composite sample. The sparse-view imaging data 2500 includes three images 2502, 2504, and 2506.

The sparse-view image 2502 of the sparse-view imaging data 2500 depicts the 4× view of the bin1 ceramic composite sample acquired using a prior method for image restoration. The second sparse-view image 2504 is acquired using an improved method that includes various trained models that the prior method lacks. The third sparse-view image 2506 is acquired using the present method. As shown in FIG. 25, the sparse-view image 2506 acquired by the present method achieves the highest quality in the overall image, e.g., having the lowest level of noise and artifacts. Additionally, the sparse-view image 2506 shows significant improvement over the sparse-view images 2502 and 2506 in micro-structures/faint structures/edges 2512 and 2514. This demonstrates the significant improvement in image quality achieved by the present synthetic prior-based method over prior approaches.

Taken as a whole, the disclosed method is a staged residual learning framework wrapped around a consistent analytical CT reconstruction front end. First, grouped full-view raw-data reconstructions are used to learn a synthetic prior model. Second, that prior is combined with a spectrally remapped difference field to construct a realistic noise training volume for the final image quality model. Third, the same principle is extended into the throughput-conditioned sparse-view continuation so that reconstruction quality and throughput adaptation are learned within one unified framework. The defining feature of the method is therefore not just deep reconstruction, but the explicit coupling of analytical reconstruction, prior learning, spectral noise modeling, and throughput-specific retraining into a single end-to-end reconstruction strategy.

The foregoing description of the embodiments, including illustrated embodiments, has been presented only for the purpose of illustration and description and is not intended to be exhaustive or limiting to the precise forms disclosed. Numerous modifications, adaptations, and uses thereof will be apparent to those skilled in the art. Numerous changes to the disclosed embodiments can be made in accordance with the disclosure herein, without departing from the spirit or scope of the disclosure. Thus, the breadth and scope of the present disclosure should not be limited by any of the above described embodiments.

Although certain aspects and features of the present disclosure have been illustrated and described with respect to one or more implementations, equivalent alterations and modifications will occur or be known to others skilled in the art upon the reading and understanding of this specification and the annexed drawings. In addition, while a particular feature may have been disclosed with respect to only one of several implementations, such feature may be combined with one or more other features of the other implementations as may be desired and advantageous for any given or particular application.

The terminology used herein is for the purpose of describing particular embodiments only, and is not intended to be limiting of the disclosure. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof, are used in either the detailed description and/or the claims, such terms are intended to be inclusive in a manner similar to the term “comprising.”

One or more elements or aspects or steps, or any portion(s) thereof, from one or more of any of the claims below can be combined with one or more elements or aspects or steps, or any portion(s) thereof, from one or more of any of the other claims below or combinations thereof, to form one or more additional implementations and/or claims of the present disclosure.

What is claimed is:

1. A computer-implemented method for reconstructing an image from sparse-view computed tomography (CT) imaging data, the method comprising:
receiving sparse-view imaging data of a subject acquired by a CT imaging device at a throughput-conditioned acquisition setting, the sparse-view imaging data having fewer projection views than full-view imaging data of the subject;
reconstructing the sparse-view imaging data using an analytical reconstruction algorithm to produce a sparse-view reconstruction;
generating a final reconstructed volume of the subject, the final reconstructed volume being generated from a trained image quality model that is created using a synthetic prior;
constructing a throughput-conditioned spectral noise field, wherein constructing the throughput-conditioned spectral noise field further includes:
computing a throughput difference field between the sparse-view reconstruction and the final reconstructed volume,
transforming the throughput difference field into a throughput frequency domain to obtain a throughput magnitude spectrum,
combining the throughput magnitude spectrum with a throughput phase field to produce a synthesized throughput frequency-domain representation, and
applying an inverse frequency-domain transform to the synthesized throughput frequency-domain representation to obtain the throughput-conditioned spectral noise field;
generating a throughput-conditioned synthesized noise volume by adding the throughput-conditioned spectral noise field to the final reconstructed volume;
training a throughput-conditioned machine learning model using the throughput-conditioned synthesized noise volume as input and the final reconstructed volume as a target; and
applying the throughput-conditioned machine learning model to the sparse-view reconstruction to generate a throughput-conditioned reconstructed volume.

2. The method of claim 1, wherein the trained image quality model is created by:
receiving the full-view imaging data of the subject acquired by the CT imaging device;
partitioning the full-view imaging data into a first projection subset and a second projection subset;
independently reconstructing the first projection subset and the second projection subset using the analytical reconstruction algorithm to produce a first grouped reconstruction and a second grouped reconstruction, respectively;
training a first machine learning model on the first grouped reconstruction and the second grouped reconstruction to learn a synthetic prior model;
applying the first machine learning model to a baseline reconstruction derived from the full-view imaging data to generate a synthetic prior;
constructing a full-view spectral noise field, wherein constructing the full-view spectral noise field includes:
computing a full-view difference field between the baseline reconstruction and the synthetic prior,
transforming the full-view difference field into a full-view frequency domain to obtain a full-view magnitude spectrum,
combining the full-view magnitude spectrum with a full-view phase field to produce a synthesized full-view frequency-domain representation frequency-domain representation, and
applying an inverse frequency-domain transform to the synthesized full-view frequency-domain representation to obtain the full-view spectral noise field;
generating a synthesized full-view noise volume by adding the full-view spectral noise field to the synthetic prior; and
training a second machine learning model using the synthesized full-view noise volume as input and the synthetic prior as a target to produce the trained image quality model.

3. The method of claim 2, further comprising:
providing a residual neural network to the first machine learning model; and
using the residual neural network, generating an output of the first machine learning model based on a learned correction, the output being expressed as ŷ=x+f_{θ}(x), wherein x is an input reconstruction and f_{θ}(x) is the learned correction.

4. The method of claim 3, wherein the residual neural network further comprises:
an initial three-dimensional convolution layer that extracts inter-slice features from a stack of adjacent input slices;
a two-dimensional encoder-decoder network that processes the extracted features at multiple resolution levels; and
a final three-dimensional output convolution layer that produces a volumetric output.

5. The method of claim 2, wherein the analytical reconstruction algorithm comprises a filtered back-projection (FBP), and wherein each of the baseline reconstruction, the first grouped reconstruction, the second grouped reconstruction, and the sparse-view reconstruction is generated using the filtered back-projection.

6. The method of claim 2, wherein constructing the full-view spectral noise field further comprises:
partitioning the full-view difference field into local volumetric chunks;
for each chunk, computing a local fast Fourier transform (FFT) to generate a local magnitude spectrum;
for each chunk, generating the full-view phase field by one of: (i) generating randomized phases using a uniform random number generator, or (ii) computing an auto-phase field derived from a frequency-domain representation of a random noise field seeded by a local spatially variant standard deviation of the full-view difference field; and
for each chunk, combining the local magnitude spectrum with the full-view phase field, and applying an inverse FFT to produce a local synthesized noise chunk.

7. The method of claim 2, wherein training the second machine learning model comprises:
computing a mean and a standard deviation of the synthesized full-view noise volume; and
normalizing both the synthesized full-view noise volume and the synthetic prior using the mean and standard deviation prior to training the second machine learning model, wherein normalized values are computed as x′=(x−μ)/σ, where μ is the mean and σ is the standard deviation of the synthesized full-view noise volume.

8. The method of claim 2, wherein training the second machine learning model further comprises:
deriving an edge mask from the synthetic prior by computing a magnitude of a Gaussian-smoothed residual of the synthetic prior; and
weighing a loss function during training, wherein regions identified by the edge mask receive higher loss weights than non-edge regions.

9. The method of claim 8, wherein deriving the edge mask further comprises restricting the edge mask to a disk-shaped support region when a circular field-of-view constraint is active.

10. The method of claim 1, wherein constructing the throughput-conditioned spectral noise field further comprises:
partitioning the throughput difference field into local volumetric chunks;
for each chunk, computing a local fast Fourier transform (FFT) to generate a local magnitude spectrum;
for each chunk, generating the throughput phase field by one of: (i) generating randomized phases using a uniform random number generator, or (ii) computing an auto-phase field derived from a frequency-domain representation of a random noise field seeded by a local spatially variant standard deviation of the throughput difference field; and
for each chunk, combining the local magnitude spectrum with the throughput phase field, and applying an inverse FFT to produce a local synthesized noise chunk.

11. The method of claim 10, wherein each local volumetric chunk has dimensions of 64×64×64 voxels, and wherein the throughput difference field is padded along a slice direction to a multiple of 64 voxels prior to partitioning.

12. The method of claim 10, further comprising rescaling the local synthesized noise chunk, wherein a standard deviation of each of the local synthesized noise chunk matches a standard deviation of a corresponding chunk of the throughput difference field.

13. The method of claim 1, wherein training the throughput-conditioned machine learning model further comprises a mean-squared error loss defined as:
$${L}_{MSE} = \frac{1}{N} {\sum }_{i = 1}^{N} {( {\hat{y}}_{i} - {y}_{i} )}^{2} ,$$
where ŷᵢ is a predicted output and yᵢ is a corresponding target.

14. The method of claim 1, the throughput-conditioned acquisition setting defines a reduction factor in a number of projection views relative to the full-view imaging data, and wherein the full-view imaging data is grouped according to the throughput-conditioned acquisition setting to select a subset of projection views corresponding to the sparse-view imaging data.

15. The method of claim 1, wherein the CT imaging device acquires the sparse-view imaging data using a three-dimensional cone-beam CT geometry.

16. The method of claim 1, wherein the subject comprises an industrial sample, and the sparse-view imaging data is acquired in a high-throughput industrial inspection setting.

17. A system for reconstructing computed tomography (CT) images from sparse-view imaging data, the system comprising:
a CT imaging device having a throughput-conditioned acquisition setting;
one or more processors; and
a memory coupled to the one or more processors and storing instructions that, when executed by the one or more processors, cause the system to:
receive sparse-view imaging data of a subject acquired by the CT imaging device at the throughput-conditioned acquisition setting, the sparse-view imaging data having fewer projection views than full-view imaging data of the subject;
reconstruct the sparse-view imaging data using an analytical reconstruction algorithm to produce a sparse-view reconstruction;
generate a final reconstructed volume of the subject, the final reconstructed volume being generated from a trained image quality model that is created using a synthetic prior;
construct a throughput-conditioned spectral noise field, wherein constructing the throughput-conditioned spectral noise field further includes:
computing a throughput difference field between the sparse-view reconstruction and the final reconstructed volume,
transforming the throughput difference field into a throughput frequency domain to obtain a throughput magnitude spectrum,
combining the throughput magnitude spectrum with a throughput phase field to produce a synthesized throughput frequency-domain representation, and
applying an inverse frequency-domain transform to the synthesized throughput frequency-domain representation to obtain the throughput-conditioned spectral noise field;
generate a throughput-conditioned synthesized noise volume by adding the throughput-conditioned spectral noise field to the final reconstructed volume;
train a throughput-conditioned machine learning model using the throughput-conditioned synthesized noise volume as input and the final reconstructed volume as a target; and
apply the throughput-conditioned machine learning model to the sparse-view reconstruction to generate a throughput-conditioned reconstructed volume.

18. The system of claim 17, wherein the instructions further cause the system to generate the final reconstructed volume by:
receiving the full-view imaging data of the subject acquired by the CT imaging device;
partitioning the full-view imaging data into two projection subsets and independently reconstructing each subset using the analytical reconstruction algorithm to produce a pair of grouped reconstructions;
training a first residual neural network on the pair of grouped reconstructions to learn a synthetic prior model;
applying the first residual neural network to a full-data reconstruction to generate the synthetic prior;
constructing a full-view spectral noise field, wherein constructing the full-view spectral noise field includes:
computing a full-view difference field between the full-data reconstruction and the synthetic prior,
transforming the full-view difference field into a frequency domain to obtain a full-view magnitude spectrum,
combining the full-view magnitude spectrum with a full-view phase field to produce a synthesized full-view frequency-domain representation,
applying an inverse frequency-domain transform to obtain the full-view spectral noise field; and
applying the trained image quality model to the full-data reconstruction to generate the final reconstructed volume.

19. A non-transitory computer-readable medium storing instructions that, when executed by one or more processors, cause the one or more processors to perform a method comprising:
receiving sparse-view imaging data of a subject acquired by a CT imaging device at a throughput-conditioned acquisition setting, the sparse-view imaging data having fewer projection views than full-view imaging data of the subject;
reconstructing the sparse-view imaging data using an analytical reconstruction algorithm to produce a sparse-view reconstruction;
generating a final reconstructed volume of the subject, the final reconstructed volume being generated from a trained image quality model that is created using a synthetic prior;
constructing a throughput-conditioned spectral noise field, wherein constructing the throughput-conditioned spectral noise field further includes:
computing a throughput difference field between the sparse-view reconstruction and the final reconstructed volume,
transforming the throughput difference field into a throughput frequency domain to obtain a throughput magnitude spectrum,
combining the throughput magnitude spectrum with a throughput phase field to produce a synthesized throughput frequency-domain representation, and
applying an inverse frequency-domain transform to the synthesized throughput frequency-domain representation to obtain the throughput-conditioned spectral noise field;
generating a throughput-conditioned synthesized noise volume by adding the throughput-conditioned spectral noise field to the final reconstructed volume;
training a throughput-conditioned machine learning model using the throughput-conditioned synthesized noise volume as input and the final reconstructed volume as a target; and
applying the throughput-conditioned machine learning model to the sparse-view reconstruction to generate a throughput-conditioned reconstructed volume.

20. The non-transitory computer-readable medium of claim 19, wherein the analytical reconstruction algorithm comprises filtered back-projection, and wherein the subject is imaged using a three-dimensional cone-beam CT geometry in an industrial or high-throughput imaging application.
